SEQUENCE LISTING (3 sequences)

SEQ ID NO: 1 - DNA, 318 bp, Artificial Sequence
  misc_feature 1..318: Sequence is fictional
cgcgatcgtatagtgcacgactgtagtcgagctaggctatacgatgtgcagcatgctagctgagcgagcgtagctagctacacgtagctaggccgcgattatatgcagctgactgtagccgatgcgagctcgccatgtagcgactgatttgcaaacgtgcgcgatcgtatagtgcacgactgtagtcgagctaggctatacgatgtgcagcatgctagctgagcgagcgtagctagctacacgyagctaggccgcgattatatgcagctgactgtagccgatgcgagctcgccatgtagcgactgatttgcaaacgtg

SEQ ID NO: 2 - DNA, 118 bp, Artificial Sequence
  misc_feature 1..118: Sequence corresponds to nucleotides 41 to 158 of SEQ ID NO. 1
acgatgtgcagcatgctagctgagcgagcgtagctagctacacgtagctaggccgcgattatatgcagctgactgtagccgatgcgagctcgccatgtagcgactgatttgcaaacgt

SEQ ID NO: 3 - DNA, 81 bp, Artificial Sequence
  misc_feature 1..81: Sequence corresponds to nucleotides 50 to 131 of SEQ ID NO. 1
ctgagcgagcgtagctagctacacgtagctaggccgcgattatatgcagctgactgtagccgatgcgagctcgccatgtag

(19) United States
(12) Patent Application Publication
(10) Pub. No.: US 2008/0281530 A1
(43) Pub. Date: Nov. 13, 2008

(54) GENOMIC DATA PROCESSING UTILIZING CORRELATION ANALYSIS OF NUCLEOTIDE LOCI

(75) Inventors: Scott A. TENENBAUM, Selkirk, NY (US); Christopher ZALESKI, Guilderland, NY (US); Francis DOYLE, Albany, NY (US); Ajish GEORGE, Timonium, MD (US)

Correspondence Address: HESLIN ROTHENBERG FARLEY & MESITI PC, 5 COLUMBIA CIRCLE, ALBANY, NY, 12203, US

(73) Assignee: The Research Foundation of State University of New York, Albany, NY (US)

(21) Appl. No.: 12/026,035

(22) Filed: Feb. 5, 2008

Related U.S. Application Data
(60) Provisional application No. 60/917,155, filed on May 10, 2007.
(60) Provisional application No. 60/975,979, filed on Sep. 28, 2007.

(51) Int. Cl. G06F 19/00; G01N 33/48

(52) U.S. Cl. 702/20

(57) ABSTRACT

Processing of genomic data is provided utilizing correlation analysis of first and second nucleotide loci employing a selected comparison type and value. The comparison type is either intersection or proximity type, and the comparison value is either a number (n) of nucleotide positions, wherein n≧1, or a percent number (pn) of nucleotide positions, wherein pn≧0, to be employed in comparing the loci. When intersection type is selected, correlation is defined by the loci overlapping with at least the number (n) of nucleotide positions in common, or by the loci overlapping with at least the percent number (pn) of nucleotide positions in common relative to a smaller one of the first and second loci, or when proximity type is selected, correlation is defined by the first and second loci being within at least the number (n) of nucleotide positions.

### CROSS-REFERENCE TO RELATED APPLICATIONS

This application claims the benefit of U.S. Provisional Application No. 60/917,155, filed May 10, 2007, entitled “System and Method for Data Retrieval and Analysis”, and U.S. Provisional Application No. 60/975,979, filed Sept. 28, 2007, entitled “Genomic Data Processing Utilizing Correlation Analysis of Nucleotide Loci”, both of which are hereby incorporated herein by reference in their entirety. In addition, this application contains subject matter which is related to the subject matter of the following applications, each of which is assigned to the same assignee as this application, and filed on the same day as this application. Each of the below-listed applications is hereby incorporated herein by reference in its entirety:
“Genomic Data Processing Utilizing Correlation Analysis of Nucleotide Loci of Multiple Data Sets”, Tenenbaum et al., Ser. No. ______, (Docket No. 0794.087B), filed herewith;
“Segmented Storage and Retrieval of Nucleotide Sequence Information”, Tenenbaum et al., Ser. No. ______, (Docket No. 0794.087C), filed herewith; and
“Non-Random Control Data Set Generation for Facilitating Genomic Data Processing”, Tenenbaum et al., Ser. No. ______, (Docket No. 0794.087D), filed herewith.

### STATEMENT REGARDING FEDERALLY SPONSORED RESEARCH

This invention was made, in part, under Grant Number 1043750 from the National Human Genome Research Institute/National Institutes of Health. Accordingly, the United States Government may have certain rights in the invention.

### TECHNICAL FIELD

This invention relates generally to processing of genomic data in the field of bio-informatics, and more particularly, to techniques for facilitating correlation analysis of nucleotide loci of one or more data sets comprising genomic data.

### BACKGROUND OF THE INVENTION

Through the use of recent technology advances, systems biology and related experiments have gained wide acceptance in the biological community. Experiments in this field result in extensive amounts of data, and very often this data represents a group or groups of polynucleotides. These polynucleotides can have many attributes, including: DNA or RNA; relative quantities; length(s); nucleotide sequence; and putative function. As a result of the human genome project, another attribute is able to be added; that is, genomic location.

Tools have been developed to visualize genomic data, using the genomic coordinates as a common thread. One example of this is the genomic browser at UCSC (http://genome.ucsc.edu/). The UCSC genome bio-informatics site acts as a central repository for data related to the human genome project, and provides a web-based visualization tool for viewing the data.

While existing tools for visualization of genomic data are vital to progress of the biological community, analysis of this data is also critical and has not been nearly as well addressed.

### SUMMARY OF THE INVENTION

Disclosed herein are a suite of data storage, retrieval, analysis and display processes and tools which focus on the genomic location attribute of data generated by, for example, systems biology experiments. Genomic location is a set of coordinates, comprising a chromosome identification, a nucleotide start position and a nucleotide end position, which represent the point of origin and position of a nucleotide locus or nucleotide sequence. This attribute is significant because it homogenizes polynucleotide data and gives a common attribute across data set instances, regardless of source. This homogizing attribute allows analysis of large amounts of data from many disparate sources and produces useful and relevant results. More particularly, presented herein is a gene regulation informatics platform actively fitted to support ongoing research in gene regulation and functional genomics. A need exists for innovative tools and resources in this area which can provide customized search, exploration, analysis and hypothesis generation. Such tools must keep pace with the dynamically changing world of gene regulation (ranging from transcriptional regulation, DNA methylation, chromatin remodeling, histone modification, post-transcriptional regulation by RNAs), as well as provide new perspectives and insights.

Thus, provided herein in one aspect, is a computer-implemented method of processing genomic data, which includes: obtaining a first nucleotide locus and a second nucleotide locus representative of genomic data mapped to a genomic coordinate system; performing correlation analysis of the first nucleotide locus and second nucleotide locus. The performing correlation analysis includes: selecting a comparison type and a comparison value for use in performing correlation analysis, the comparison type comprising one of intersection type or proximity type, and the comparison value comprising a number (n) of nucleotide positions, wherein n≧1, or a percent number (pn) of nucleotide positions, wherein pn≧0, to be employed in determining whether the first nucleotide locus and the second nucleotide locus correlate; and comparing the first and second nucleotide loci for correlation utilizing the selected comparison type and comparison value, wherein when intersection type is selected, and depending on the selected comparison value, correlation is defined by the first nucleotide locus and the second nucleotide locus overlapping with at least the number (n) of nucleotide positions in common, or by the first nucleotide locus and the second nucleotide locus overlapping with at least the percent number (pn) of nucleotide positions in common relative to a smaller one of the first nucleotide locus and the second nucleotide locus, or when proximity type is selected, correlation is defined by the first nucleotide locus and the second nucleotide locus being within at least the number (n) of nucleotide positions. Once correlation analysis is performed, the method further includes outputting results of the correlation analysis of the first and second nucleotide loci.

In another aspect, a system for processing genomic data is provided. The system includes memory for holding a first nucleotide locus and a second nucleotide locus representative of genomic data mapped to a genomic coordinate system, as well as a correlation analysis tool to perform correlation analysis on the first nucleotide locus and the second nucleotide locus. The correlation analysis tool includes select logic and comparison logic. The select logic allows designation of a comparison type and a comparison value to be used by the correlation analysis tool in performing the correlation analysis. The comparison type includes one of intersection type or proximity type, and the comparison value includes a number (n) of nucleotide positions, wherein n≧1, or a percent number (pn) of nucleotide positions, wherein pn≧0, to be employed in determining whether the first nucleotide locus and the second nucleotide locus correlate. The comparison logic is employed to determined whether the first and second nucleotide loci correlate, and utilizes the selected comparison type and comparison value in performing the correlation analysis. When intersection type is selected, and dependent on the correlation value selected, correlation is defined by the first nucleotide locus and the second nucleotide locus overlapping with at least the number (n) of nucleotide positions in common, or by the first nucleotide locus and the second nucleotide locus overlapping with at least the percent number (pn) of nucleotide positions in common relative to a smaller one of the first nucleotide locus and the second nucleotide locus. When proximity type is selected, correlation is defined by the first nucleotide locus and the second nucleotide locus being within at least the number (n) of nucleotide positions. The system further includes output logic to provide results of the correlation analysis of the first nucleotide locus and the second nucleotide locus.

In a further aspect, an article of manufacture is provided which includes at least one computer-usable storage device having computer-readable program code logic to facilitate processing of genomic data. The computer-readable program code logic when executing performs the following: obtaining a first nucleotide locus and a second nucleotide locus representative of genomic data mapped to a genomic coordinate system; performing correlation analysis on the first nucleotide locus and the second nucleotide locus; and outputting results of the correlation analysis of the first and second nucleotide loci. The performing correlation analysis includes: selecting a comparison type and a comparison value for use in performing the correlation analysis, the comparison type comprising one of intersection type or proximity type, and the comparison value comprising a number (n) of nucleotide positions, wherein n≧1, or a percentage number (pn) of nucleotide positions, wherein pn≧0, to be employed in determining whether the first nucleotide locus and the second nucleotide locus correlate; and comparing the first and second nucleotide loci for correlation utilizing the selected comparison type and comparison value, wherein when intersection type is selected, and dependent on the correlation value selected, correlation is defined by the first nucleotide locus and the second nucleotide locus overlapping with at least the number (n) of nucleotide positions in common, or by the first nucleotide locus and the second nucleotide locus overlapping with at least the percent number (pn) of nucleotide positions in common relative to a smaller one of the first nucleotide locus and the second nucleotide locus, or when proximity type is selected, correlation is defined by the first nucleotide locus and the second nucleotide locus being within at least the number (n) of nucleotide positions.

Further, additional features and advantages are realized through the techniques of the present invention. Other embodiments and aspects of the invention are described in detail herein and are considered a part of the claimed invention.

### BRIEF DESCRIPTION OF THE DRAWINGS

The subject matter which is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 is a partial depiction of a conventional genomic browser display showing a portion of the human genome with multiple data sets displayed;

FIG. 2 depicts one embodiment of a system for processing genomic data, in accordance with one or more aspects of the present invention;

FIG. 3A depicts one embodiment of logic for performing correlation analysis of a mapped experimental data set and at least one other mapped data set, in accordance with an aspect of the present invention;

FIG. 3B depicts an alternate embodiment of logic for performing correlation analysis of a mapped experimental data set and at least one other mapped data set, in accordance with one or more aspects of the present invention;

FIG. 4 depicts one embodiment of logic for processing genomic data using the system and tools of FIG. 2, in accordance with one or more aspects of the present invention;

FIG. 5 depicts a database schema for facilitating storage of different types of genomic data and providing access thereto, in accordance with one or more aspects of the present invention;

FIG. 6 illustrates transformation of an experimental data set into a data model comprising a locus set object and multiple locus objects for facilitating analysis and manipulation of the data set, in accordance with one or more aspects of the present invention;

FIG. 7 depicts one embodiment of logic for facilitating transformation of genomic data into mapped genomic data, in accordance with one or more aspects of the present invention;

FIG. 8 is an example of transformation of genomic data visualized in the browser depiction of FIG. 1 utilizing the data model transformation processing of FIGS. 6 & 7, in accordance with one or more aspects of the present invention;

FIG. 9 depicts one embodiment of logic for adding a genomic sequence to a segmented sequence table of a database structured as disclosed herein, in accordance with one or more aspects of the present invention;

FIG. 10 depicts one embodiment of logic for retrieving a genomic sequence from a segmented sequence table of a database structured as disclosed herein, in accordance with one or more aspects of the present invention;

FIGS. 11A-11C illustrate sequence storage into and retrieval from a segmented sequence table, in accordance with one or more aspects of the present invention;

FIG. 12 depicts one embodiment of logic for sorting locus objects, in accordance with one or more aspects of the present invention;

FIG. 13 depicts one embodiment of logic for performing correlation analysis of nucleotide loci, in accordance with one or more aspects of the present invention;

FIG. 14 depicts one embodiment of logic for compressing nucleotide loci, for example, within a locus set object, in accordance with one or more aspects of the present invention;

FIG. 15A depicts an example of nucleotide loci (or locus objects) to undergo correlation analysis for compression within three locus set objects (i.e., Set A, Set B & Set C), in accordance with one or more aspects of the present invention;

FIG. 15B depicts the locus set objects of FIG. 15A, after the nucleotide loci within each locus set object have been compressed, in accordance with one or more aspects of the present invention;

FIG. 16 depicts one embodiment of logic for user-defining of parameters employed in non-randomly generating a control data set, in accordance with one or more aspects of the present invention;

FIG. 17 depicts one embodiment of logic for non-randomly generating a control data set, in accordance with one or more aspects of the present invention;

FIGS. 18A & 18B graphically depict an example of updating of a selected set of nucleotide regions for analysis from three locus set objects undergoing correlation analysis, in accordance with one or more aspects of the present invention;

FIG. 19A depicts the three original locus set objects of FIG. 15A, to undergo correlation analysis and data structure definition, in accordance with one or more aspects of the present invention;

FIG. 19B displays results of correlation analysis and data structure definition for the three data set example of FIG. 19A, wherein the data structure includes a union locus, all original nucleotide loci which correlate, and an intersection locus, where correlation is defined by a minimum of one nucleotide position overlap and bridging between nucleotide loci is false (i.e., not considered), in accordance with one or more aspects of the present invention;

FIG. 19C displays alternate results of correlation analysis and data structure definition for the three data set example of FIG. 19A, wherein a different data structure is defined, including all original nucleotide loci which correlate, a union locus, and an intersection locus, which result when correlation is defined by a minimum of one nucleotide position overlap and bridging between nucleotide loci of the locus set objects is true (i.e., considered), in accordance with one or more aspects of the present invention.

FIG. 20 depicts one embodiment of logic for performing correlation analysis of nucleotide regions across multiple data sets, in accordance with one or more aspects of the present invention;

FIG. 21 depicts one embodiment of logic for aggregating negative locus set objects, sorting nucleotide loci within a locus set object, and compressing nucleotide loci to define nucleotide regions to be employed by the logic of FIG. 20, in accordance with one or more aspects of the present invention;

FIG. 22 depicts one embodiment of logic for aggregating correlated nucleotide loci into a data structure comprising a union locus, in accordance with one or more aspects of the present invention;

FIG. 23 depicts one embodiment of logic for updating a selected set of nucleotide regions from multiple data sets (or locus set objects) undergoing correlation analysis, in accordance with one or more aspects of the present invention;

FIG. 24 depicts one embodiment of logic for determining whether correlated nucleotide regions overlap with one or more negative regions of the aggregate negative locus set, in accordance with one or more aspects of the present invention;

FIG. 25 depicts one embodiment of a flow diagram comprising an interactive display of mapped data sets and session states for a plurality of mapped data sets undergoing control data set generation and correlation analysis, in accordance with one or more aspects of the present invention; and

FIG. 26 depicts one embodiment of a computer program product to incorporate one or more aspects of the present invention.

### DETAILED DESCRIPTION OF THE INVENTION

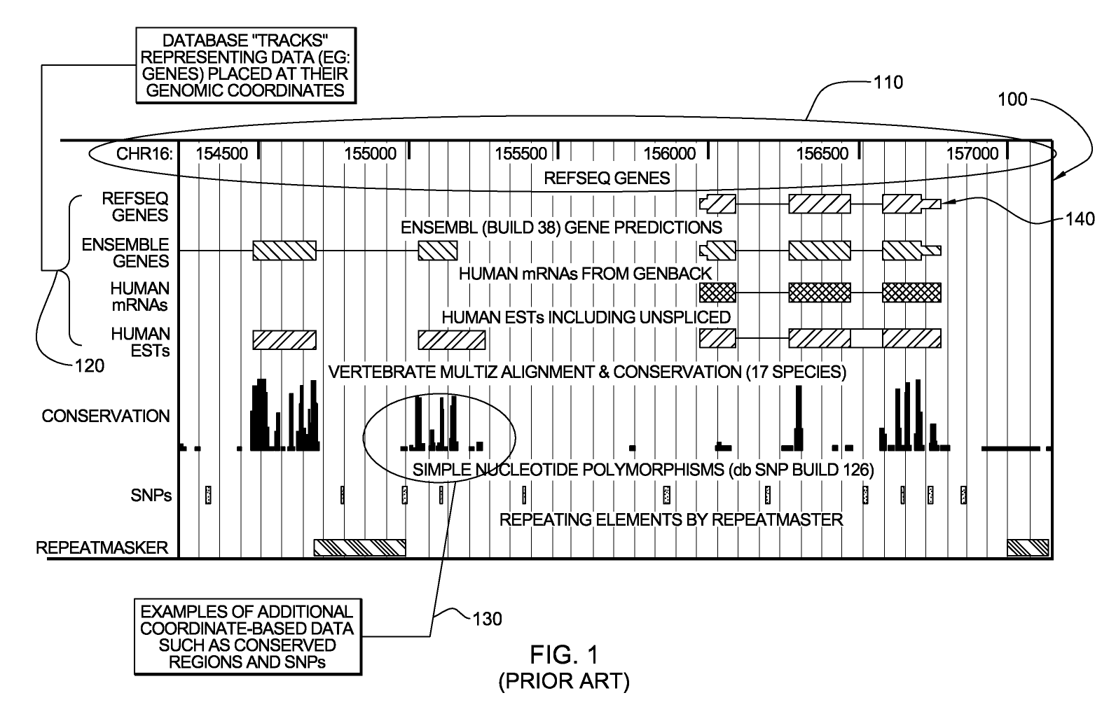

By way of example, FIG. 1 represents a UCSC genomic browser display, generally denoted 100, illustrating a portion of the human genome with multiple existing data sets 120, 130 superimposed thereon. In the UCSC genomic browser, chromosomes are displayed in linear fashion from left to right, with coordinate markers 110 appearing across the top as illustrated. In this example, nucleotide positions 154000-157000 are illustrated for chromosome 16. Data sets 120, such as genes, are shown in a similar manner, with each item displayed at its appropriate coordinates. Multiple data sets are shown simultaneously by stacking the data sets 120, 130 from top to bottom. The view can be scaled to various levels of “zoom”, but in order to view relevance, one must scale the view to an extremely small portion of the total chromosome. Thus, only a minute portion of the data can be visually analyzed at any one time using the UCSC genomic browser. In the example illustrated, ReqSeq Genes, Ensemble Genes, Human mRNAs, Human ESTs, Conservation, SNPs, and Repeatmasker data sets are illustrated. Data 140 is an example of a single data record, which in this example represents a gene. Although powerful as a visualization tool, the UCSC genomic browser is less helpful in terms of analysis of the genomic data.

Presented herein are various techniques for processing and analysis of genomic data in the field of bio-informatics. More particularly, a suite of data retrieval and analysis tools and processes are disclosed which focus on the genomic coordinate attribute of genomic data generated, for example, by systems biology experiments. This homogizing attribute allows for analysis of large amounts of information from many disparate sources, while producing useful and relevant results.

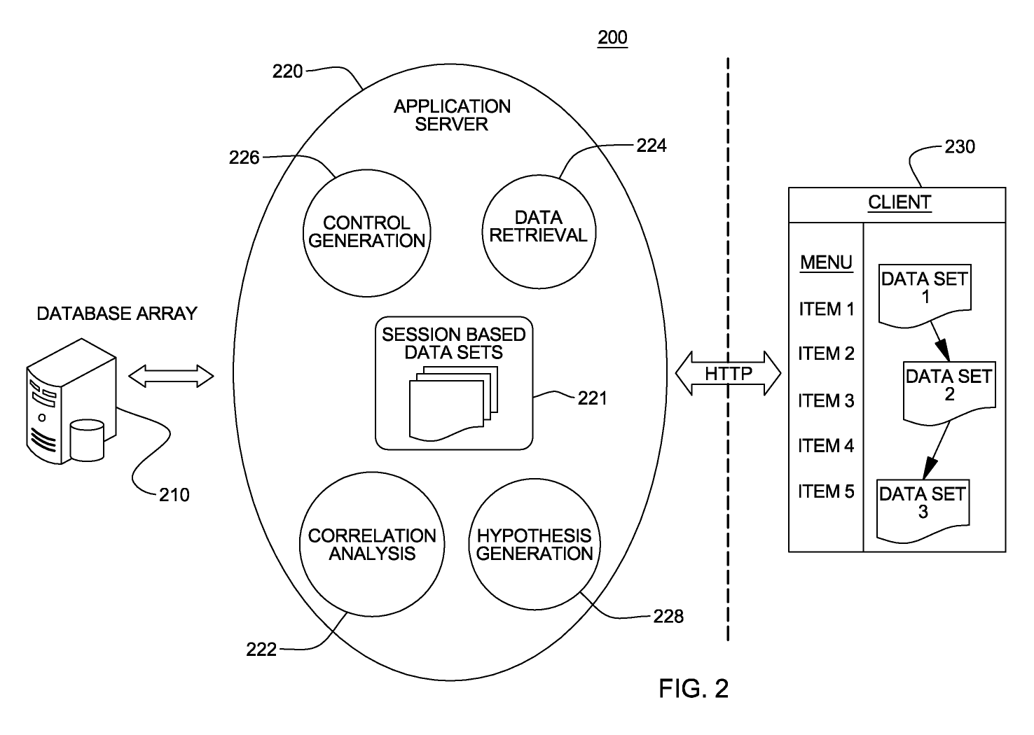

FIG. 2 illustrates one embodiment of a system, generally denoted 200, for processing genomic data in accordance with one or more aspects of the invention disclosed herein. In this example, system 200 is a three-tier system utilizing a relational database array 210, a web-based application server 220, and one or more web browser clients 230. The three-tier system 200 of FIG. 2 is presented by way of example only. In other implementations, the concepts presented herein could be implemented in alternate computing configurations, including as a stand-alone workstation.

Relational database array 210 may be implemented using, for example, MySQL, version 5, offered by My SQL AB (httn://www.mysql.com/company/). The databases within relational database array 210, which are each contextual in one embodiment to a species and assembly (described further below), may reside within a single instance of the database engine. This instance can reside at any location that is network accessible from the application server. A JDBC connection may be used to link the application server to the database. (JDBC is a Sun Microsystems standard defining how JAVA applications access database data.) As explained further below, a sub-system database manager module may be provided within relational database array 210 to facilitate access to databases from the application server. This provides a single point of access and control over the database processes.

Application server 220 may be implemented using standard J2EE technologies (servlets and JPSs) on Jakarta Tomcat, Version 5, provided by The Apache Software Foundation (http://www.apache.org/). User interaction is session-based. However, it is also possible to store a session state at the server for later retrieval. A “model-view-controller” design may be used to control interaction and data flow within the system. The model is the current set of data and state information for a user session. As described further below, it is made of locus set objects representing user-loaded and pre-existing data sets, as well as new data sets 221 generated during the session. The model also holds session state information, such as logic parameters and process cardinality. The controllers are the individual system tools which act as independent modules within the system. In this example, these modules or tools include a correlation analysis tool 222, a data retrieval tool 224, a control generation tool 226 and a hypothesis generation tool 228. Each modular tool represents a logic implementation (described below), which can execute individually or in succession.

Client 230 includes a display window illustrating the data sets and session states utilized by the client. As described below, the display window may illustrate a flow diagram which contains: data sets and their annotation; instances of modules used to process the data, along with the parameters used; and relationships among the data sets and processes describing the interactions. Further, the client is presented with a menu of operations which can be performed, such as uploading data, retrieving additional data from a database, or executing an analysis process on the data. There is also a section in the interface for user input which may be required for a given operation. This area may be contact sensitive, and present appropriate options for a currently selected operation. As noted, this is in addition to the client interface presenting the user with a view of their data and operations performed. This data and operations information is rendered as a flow diagram, sequentially describing (for example) each data set and the operations that were performed thereon. The client interface is configured such that the user can interact with the diagram to obtain more detailed information about any of the elements, download data sets, or to generate an image file for documentation purposes.

In order to utilize the processing and system capabilities disclosed herein, a data file must first contain the genomic coordinate attribute. This attribute often exists by default as part of the result of an experiment. However, the feature may not be implicit for certain technologies. For example, certain micro-array results may provide accession numbers only, or require statistical analysis before coordinates can be generated. In these cases, the system can provide a means to transform the data. For example, the database manager can be used to perform simple data look-up, such as mapping accession numbers to loci, or third party tools can be integrated into the system (such as Bioconductor (http://www.biocondutor.org/) or TileMap (http://www.bioinformatics.oxfordjournals.org/cgi/content/abstract/21/18/3629)) or the system could “link out” to a third part website service for data conversion (such as offered by NetAffx (http://www.affymetrix.com/analysis/index.affx) or TileScope (http://www.tilescope/gersteinlab.org/)).

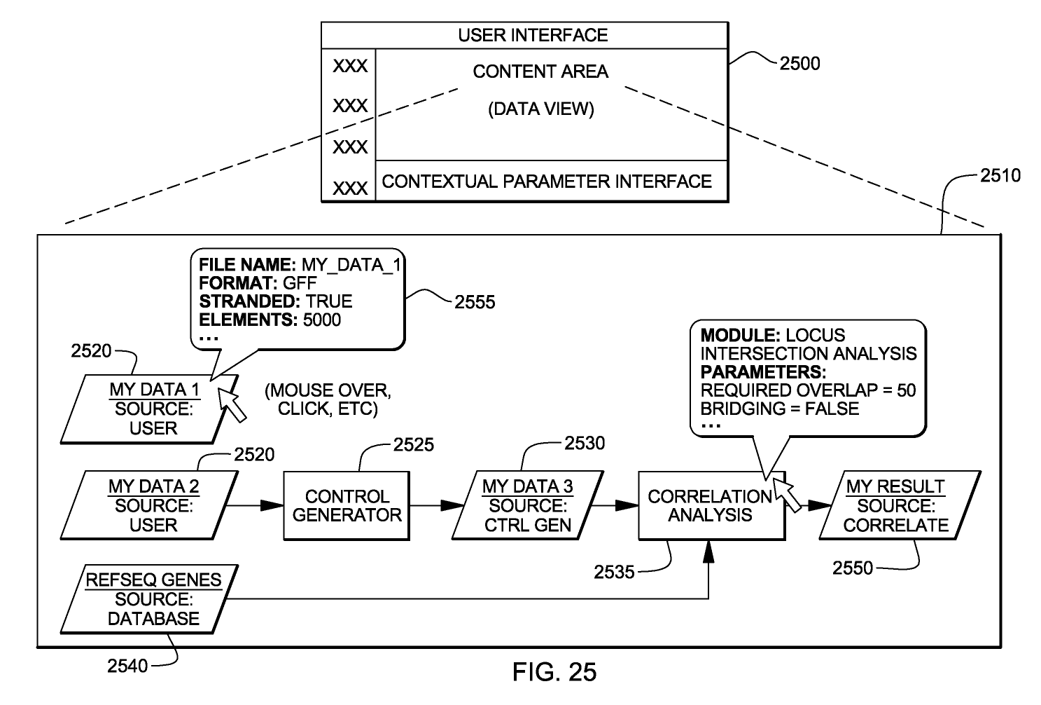

Once a data set contains genomic coordinates, it is then loaded into the system. Additional data sets can be added, for example, from the existing relational database array as desired. The user then chooses which operations are to be performed on which data sets, and resultant data sets are generated. Since all data sets are homogenous, they can be mixed and matched in any operation and in any order. The sequence of operations, data sets generated, parameters used, and all other corresponding information may be displayed in the client's flow diagram. The user can continue to perform analysis until the desired result(s) and data set(s) are generated. An example of a resultant flow diagram is presented in FIG. 25. The illustrated flow diagram presents one example of a convenient approach to view current data sets, processes performed on those data sets, and accompanying process parameters and session history information.

To summarize, the client may advantageously be designed to be runable from any web browser, and present a user with their data sets modeled in the above-described workflow diagram, as well as a “tool set” reflecting the executable modules within the system. The application server contains the user's session-based data and process state. Further, the application server may execute instances of analysis modules, manipulating the current data sets and user-defined parameters. As noted above, and described further below, the relational database array houses local instances of species and assembly genomes and associated annotations. The system depicted in FIG. 2 may also allow for optional distributed processing to ease execution of resource intensive analysis.

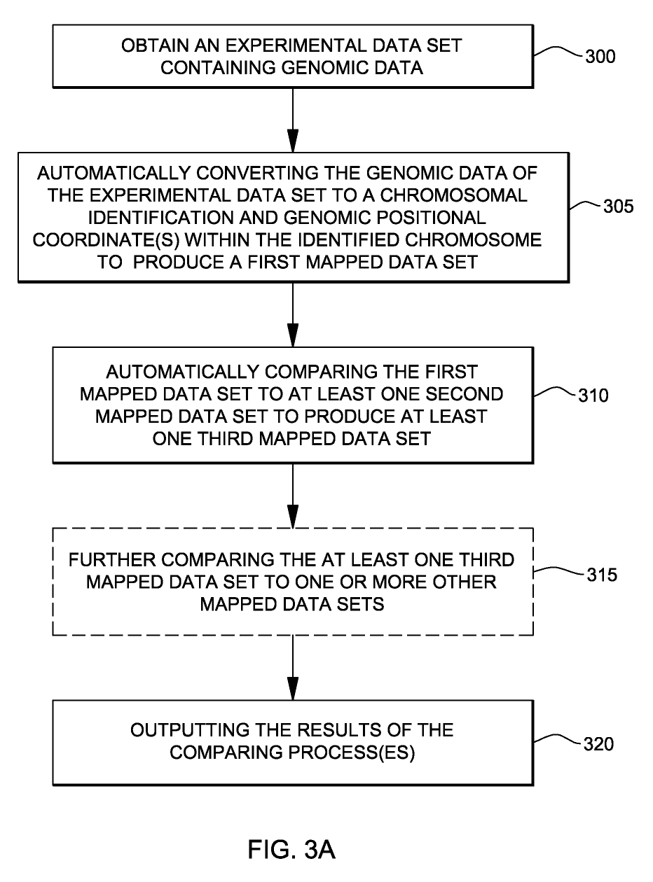

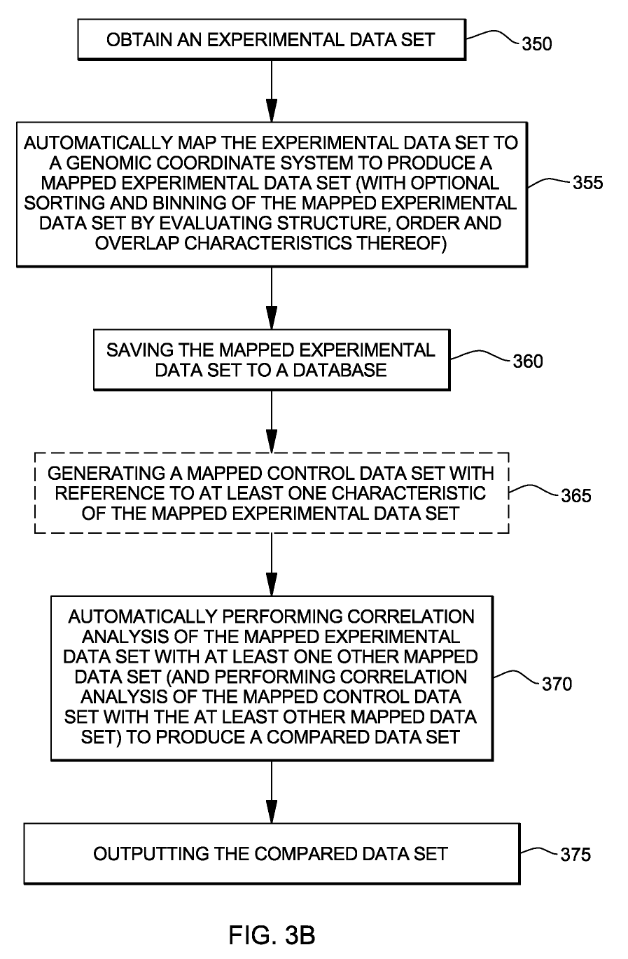

FIGS. 3A & 3B depict high level implementations of the processing disclosed herein. In FIG. 3A, an experimental data set is obtained containing genomic data 300. If not already mapped to the genomic coordinate system, then the genomic data is converted to one or more chromosomal identifications and genomic positional coordinates within the identified chromosome(s) to produce a first mapped data set 305. Thereafter, the first mapped data set is compared to at least one second mapped data set to produce at least one third mapped data set 310. This process may be repeated by comparing the at least one third mapped data set with one or more other mapped data sets in a parallel or sequential manner 315. Results of the comparing process(es) are then output 320. As used herein, “output” or “outputting” refers to displaying, printing, saving or otherwise providing or recording results of the comparing process, either for user information or for further processing, in accordance with the concepts disclosed herein.

In FIG. 3B, an experimental data set is again obtained for processing 350. As used herein, the term “obtaining” includes, but is not limited to, fetching, receiving, having, providing, being provided, creating, developing, etc. If not already containing genomic coordinates, the experimental data set is again mapped to a genomic coordinate system to produce a mapped experimental data set 355. This mapped experimental data set may also undergo optional sorting and binning of the mapped experimental data by evaluating structure, order and overlap characteristics thereof (as disclosed further herein). The mapped experimental data set is saved, in one embodiment, to a database 360. Alternatively, the mapped experimental data set could remain as session data within, for example, memory of the application server.

Optionally, a mapped control data set may be generated with reference to one or more characteristics of the mapped experimental data set 365, and in the embodiments disclosed herein, with reference to multiple characteristics thereof. Correlation analysis may be automatically performed on the mapped experimental data set with at least one other mapped data set, for example, retrieved from the relational database array 370. The result is a compared data set which is then output 375. In addition to performing correlation analysis on the mapped experimental data set with the at least one other mapped data set, correlation analysis of the mapped control data set (if created) may also be automatically performed with reference to the at least one other mapped data set, again with the results of the comparing process being output.

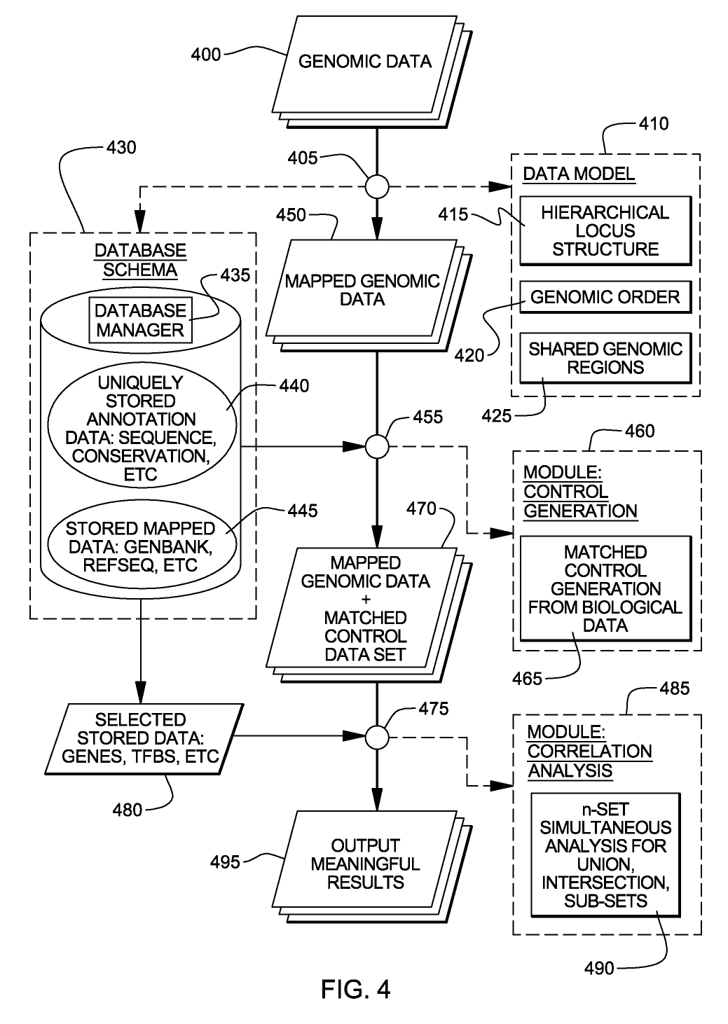

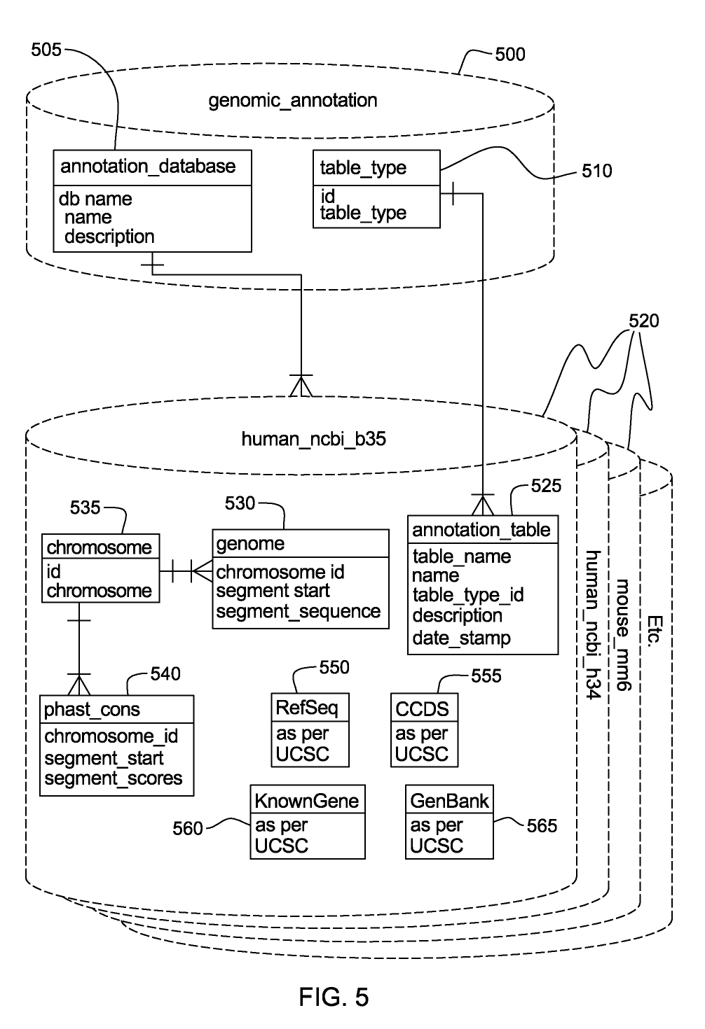

FIG. 4 depicts a further exemplary data process flow and various tools described herein used during the process flow. This diagram is presented by way of example only. In the figure, genomic data 400 is obtained, and assuming that the data is not already mapped to the genomic coordinate system, the data undergoes transformation to a mapped data set containing genomic coordinates (as introduced above and described further below). This mapping 405 results in mapped genomic data 450. The mapping process is with reference to a data model 410, also described further below. Data model 410 includes a hierarchical locus structure 415, a genomic ordering function, and a shared genomic regions compression function 425. If desired, the mapped genomic data 450 may be saved to a database 430 which includes a database manager 435 and, in this embodiment, uniquely stored annotation data (such as sequence, conservation, etc.) 440, as well as stored mapped data (such as GenBank, RefSeq, etc.) 445. The database schema for database 430 is described further below with reference to FIG. 5.

In the process flow example of FIG. 4, the mapped genomic data is employed to generate a control data set 455. This control data set generation uses a control generation tool 460 provided as part of the system disclosed herein (see FIG. 2). In particular, a matched control generation process may be used to provide a mapped control data set from multiple characteristics or attributes, for example, of the originally received experimental genomic data 400. By matching the control data set to characteristics of the mapped genomic data set, improved results are obtained when analyzing the resultant compared data sets. Output from control generation processing 455 is the mapped genomic data set and matched control data set 470. In this example, the two data sets then separately undergo correlation analysis 475 to a further selected mapped data set using a correlation analysis tool 485 of the system. In one example, the correlation analysis tool provides an n-set, simultaneous analysis for union and intersection sub-sets 490. When performing correlation analysis, selected stored data sets, such as genes, TFBS, etc., may be employed in performing the correlation analysis 475. In this example, the mapped genomic data set undergoes correlation analysis to the selected mapped data set (for example, retrieved from database 430), and the matched control data set also undergoes correlation analysis to the selected mapped data set. This results in meaningful results being obtained and output 495.

### Database Schema and Data Model:

As noted briefly above, data can originate from a variety of sources. Besides the user's own data, another source of data is pre-existing databases. For example, the system disclosed herein may maintain its own database array for: providing a local, fast look-up of common data sets for user retrieval without having to depend on third party sources; and providing specially structured and accessed database tables of additional annotation, which allow a user to rapidly recover certain additional data that is normally slow and resource-intensive to generate.

As illustrated in the database example of FIG. 5, in one embodiment the database array may be structured in a hierarchical fashion, based on genomic species and assembly (i.e., version of a genome sequence). For particular species and assemblies, there will be a number of data sets available. Much of the actual data itself may be derived directly from the UCSC website, matching table schema, indexing and content. Additional third party data sources may be leveraged as well. This allows for ease of portability and maintenance, and allows for a local copy of this data to be present. However, the database array contains a number of additional attributes which add to the functionality of the system.

The database schema depicted in FIG. 5 includes, for example, a genomic_annotation database 500 which acts as a central point of access and contains meta-data tables 505, 510 describing what information is available and how it is structured in the balance of the array. This database 500 may be used to discover what species and assembly table combinations are available, how to access those tables, as well as global table structure descriptions for each unique set of content. Specifically, tables 505, 510 in the main database 500 list what combinations are available. For example, annotation_database 505 includes database name and description for each database, and table_type 510 includes an ID and table_type for various tables 525 contained within the database array. There also exists a separate database 520 to house each species/assembly combination, and any data corresponding to a particular species/assembly combination that exists.

Advantageously, the meta-data tables 505, 510 may be employed to add new data sets to the system on the fly, and have those data sets immediately available. In addition, uniquely structured tables of additional annotation are provided which allow for rapid retrieval of large repositories of information with minimal overhead.

The database manager utilizes database 500, as well as the databases and tables therein, and takes advantage of the schema depicted in FIG. 5, as described herein. The database manager not only allows programmatic access to the data, but provides additional functionality to assist in the transformation of genetic data (e.g., genes, sequences, etc.) into mapped genomic data (i.e., coordinate-based data).

The database manager provides a list of species and assembly combinations that are available, and the user makes the appropriate choice. For the given species/assembly, a list of annotation sets are provided and the user chooses which sets are to be searched. For example, RefSeq 550, CCDS 555, KnownGene 560, and GenBank 565 may be included. If available, the database manager provides a list of sub-types called “locus types” (described further below), from which the user can choose to refine the results. If the selected annotation set represents genes, locus types could be exons, UTRs, etc. If the selected annotation set represents promoters, then the available locus type would be the entire locus. The user's accession numbers can be searched in the database, and all found items transformed into mapped coordinate-based data. Any accession numbers that could not be found would be reported back to the user.

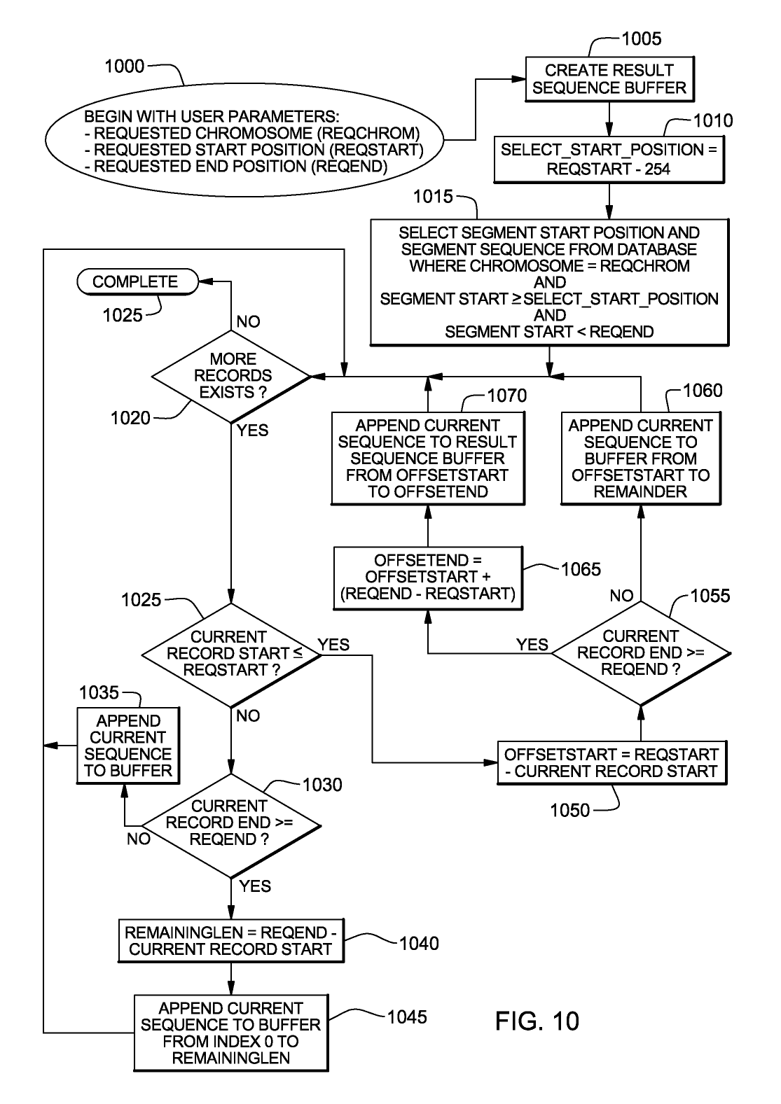

As noted, each species/assembly database thus contains a number of data sets gathered from third party sources such as UCSC or others. When describing this data, the genomic location attribute (chromosomal identifier and nucleotide coordinates) is the focus of the system described herein. However, there are other attributes of significance, such as sequence, which may be part of the analysis. Thus, the database array also provides a means by which this information can quickly and easily accompany the loci in a data set. For example, additional annotation sets may include nucleotide sequences, and phylogenetic conservation (i.e., genome table 530 and PHAST_CONS table 540, respectively). In each case, an attribute of each nucleotide must be maintained, that is, a sequence “letter” (ATCG, etc.), or a conservation score. Each table is structured in a similar manner. In particular, and as described in detail below, the attributes of each nucleotide sequence may be grouped together into equal length short segments, and each segment given its own corresponding chromosomal position. In this case, only the chromosome and first nucleotide (start position) need be tracked. An index is also created based on the chromosomal coordinates, thus giving a unique index. In this way, data that was previously “horizontal” (e.g., an entire chromosome sequence) is transformed into readily indexible, vertical data. This allows extremely fast retrieval of large amounts of information using the processing described below (for example, with reference to FIG. 10). Advantageously, this allows elimination of any seek time bottleneck, while allowing the benefits of storing raw data in a relational database. In addition to the above-noted tables, the database further includes a “chromosome” table 535, which is a normalization table which maps different nomenclature for chromosomes to a common integer element. This table facilitates data retrieval. For example, “chromosome 1”=“CHR1,”=1, “chromosome 2”=2, . . . “chromosome X”=“CHRX”=23.

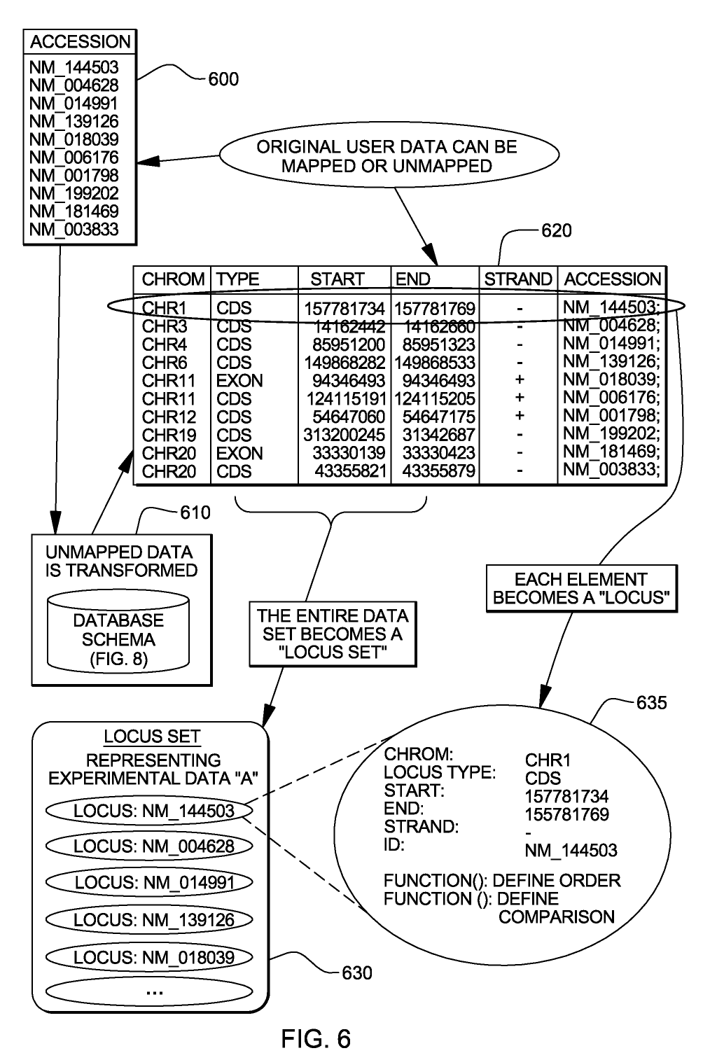

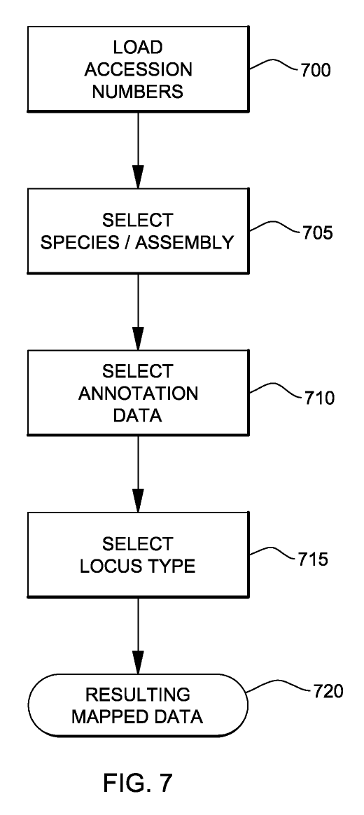

FIG. 6 illustrates an example of transformation of a list of accession/ID numbers into mapped data, in accordance with this disclosure. The accession numbers 600 represent original user unmapped data, while the data in table 620 represents original user mapped data. If unmapped, then the data is transformed for storage into the above-described database schema 610. As shown in FIG. 7, this transformation includes, for example, using the database manager to transform genetic data 600 to mapped genomic data 620. The user first loads a list of accession numbers 700 into the system, then selects the appropriate species/assembly 705 database and the appropriate annotation data 710 to be searched. An example might be human_build_{—}35−GenBank & RefSeq. If available, the user selects locus “types” they'd like to retrieve (e.g., exons, UTRs, etc.), and the accession numbers are looked-up and transformed into mapped genomic data 720. This transformed or mapped data set 620 (FIG. 6) is then modeled as a locus set object 630 and locus object 635 for analysis and manipulation, as described herein.

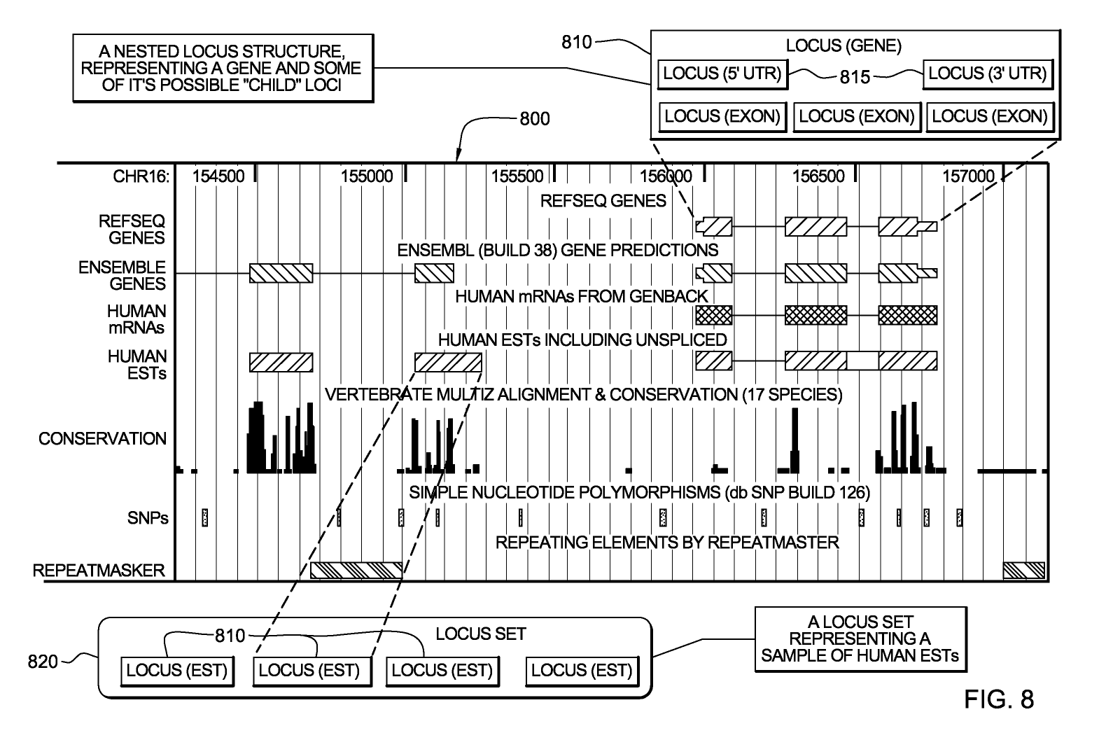

The data model disclosed herein can be better understood with reference to FIG. 8. As noted, data can originate from a variety of sources, including user-loaded data (such as the result of a micro-array experiment), pre-existing mapped data maintained in the relational database of the system, and pre-existing data from third party databases (accessed independently by the user or via a system connector). Data loaded into the system is converted into a homogenous data structure, shared by all parts of the system. This data structure is modeled in an object-oriented approach, and includes two core components; namely, locus objects and locus set objects. Each of these is constructed with its own set of attributes and built-in functionality. The attributes and functionality of these objects are as follows:

### Locus Objects:

Attributes:
A locus object includes a nucleotide locus, which is the base unit of analyzable data in the system. A nucleotide locus comprises one nucleotide position or two or more contiguous nucleotide positions.
The only required attributes are the genomic coordinates.
Remaining core attributes are modeled after the GFF specification (http://www.sanger.ac.uk/software/formats/gff/).
Any additional attributes can be added dynamically.
Locus objects have the ability to be nested in parent/child relationships.

Functionality:
Locus objects include sort logic by which they can be sorted. Sorting is contextual to their coordinate system (chromosome and position).
Locus objects also include compare logic by which they can be compared. Comparisons are contextual to their coordinate system, and result in “Before”, “After”, or “Correlate” indications.

### Locus Set Object:

Attributes:
Locus set objects are containers for grouping locus objects.
Locus set objects most often represent an experiment result file, an annotation data set, or other aggregation of genomic loci.

Functionality:
Locus set objects can be dynamically allocated and altered.
Locus set objects can be merged.
Locus set objects can effect their contained locus objects in a global manner, such as sorting or compressing.
Locus set objects include compress logic to compress correlated loci therein into regions.

Locus sorting can be accomplished using the specification for object sorting. The locus object fulfills the specification requirement by implementing a “compare to” function. Simple conditional logic can be used to perform a lexicographic comparison of chromosome values and numeric comparison of start position values.

In the example of FIG. 8, a partial display of the UCSC browser 800 is repeated, with a locus object 810 (gene) being superimposed as illustrated. Within this locus object 810, a plurality of other locus objects 815 are disposed, representing the locus gene. Thus, locus object 810 is a nested locus structure, representing (in one example) a gene and certain ones of its possible “child” loci. The locus type in this example would either be gene, 5′ UTR, 3′ UTR, or EXON. Additionally, FIG. 8 represents a locus set object 820, which is a collection of locus objects 810 relating, in one example, to a sample of human ESTs.

Returning to FIG. 6, each element in the mapped data set becomes a locus object 635, which includes the chromosome identifier, type, start and end coordinates (defining a nucleotide locus), and includes the above-noted logic functions to facilitate ordering and comparison of locus objects. Additionally, the entire mapped data set 620 becomes a locus set object 630, which includes each of the elements of the mapped data set as a separate locus object, as well as logic to facilitate compression of locus objects within the set.

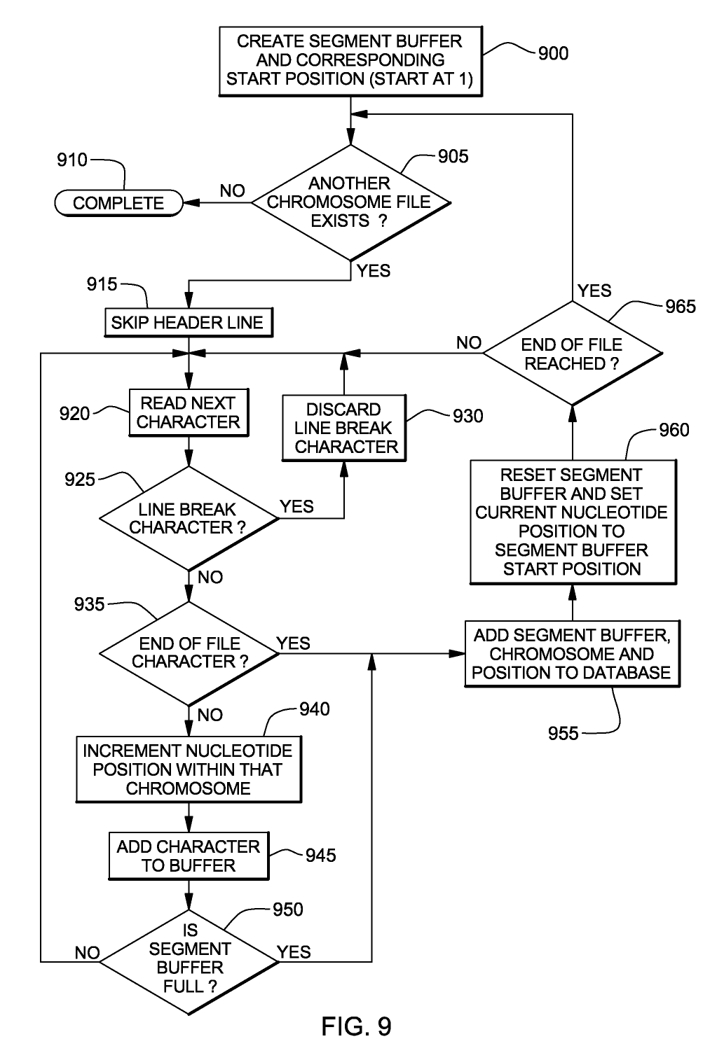

FIGS. 9-11C illustrate system logic for adding and retrieving a genomic sequence to/from a database, such as database 520 of FIG. 5.

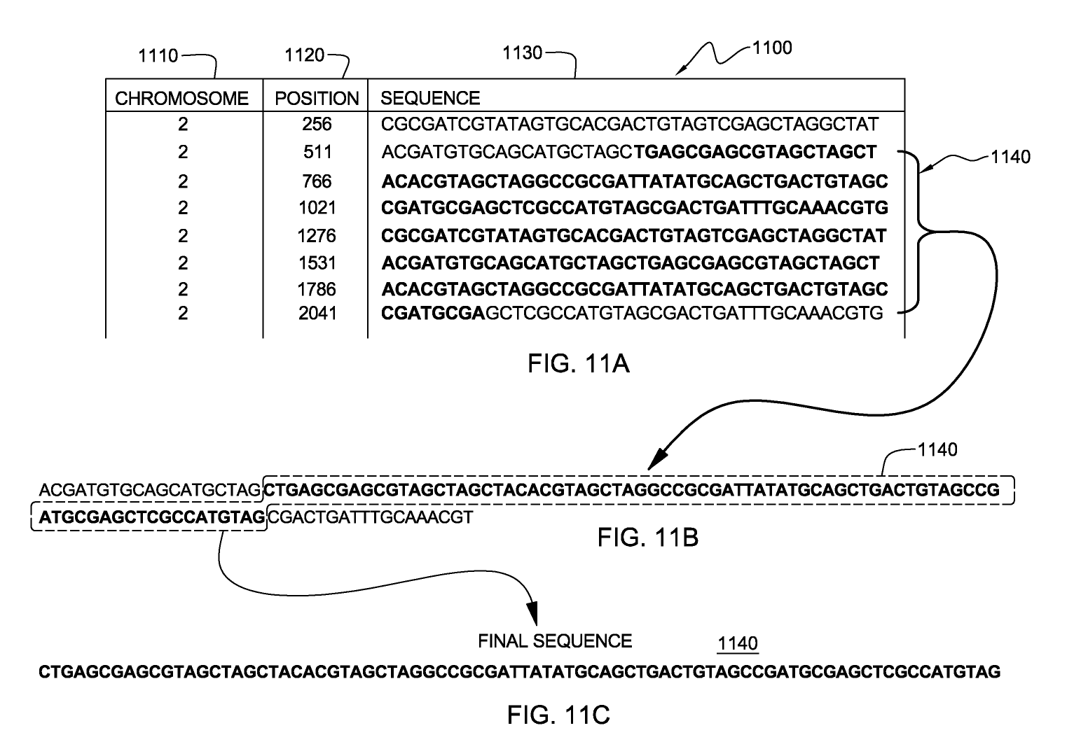

Beginning with the logic of FIG. 9, a genomic sequence may be automatically added to the database described herein by initially creating a segment buffer and identifying a corresponding start position (e.g., position 1) 900. Processing then determines whether another chromosome file exists 905, and if “no”, the process is complete 910. Assuming “yes”, then the header line for the chromosome file is skipped 915, and a next character in the file is read 920. Processing determines whether this next character is a line break character 925, and if so, the line break character is discarded 930 and a next character 920 is read. If the read character is other than a line break character, processing determines whether the character is an end of file character 935. If “no”, then the nucleotide position within that chromosome is incremented 940 and the character is added to the segment buffer 945. Processing determines whether the segment buffer is full 950. If “no”, then the next character is read 920. If the character is an end of file character, or if the segment buffer is full, then processing adds the segment buffer content, the chromosome identifier and the start position identifier to a segmented sequence table within the database 955. An example of this table is illustrated in FIG. 11A, wherein table 1100 includes a chromosome identifier 1110, a start position identifier 1120, and a sequence segment 1130 for each of a plurality of segments.

Continuing with the processing of FIG. 9, the segment buffer is reset, and the current nucleotide position is set to the segment buffer start position 960. Processing determines whether an end of file has been reached 965, and if “no”, then the next character is read 920. Otherwise, processing determines whether another chromosome file exists 905, and dependent upon on the answer, repeats as described above.

Those skilled in the art will note from the above discussion that the logic presented iterates over provided a chromosome file reading one character at a time, with each segment of characters being of a common specific size and being sequentially added to the segmented sequence table within the database. In this example, the common specific size is 255, however, other segments sizes could be employed. The chromosome and coordinate positions of each segment are also tracked and added to the database automatically.

FIGS. 10 & 11A-11C illustrate an examplary data retrieval process from a genomic sequence table, such as described above. Processing begins with user-inputted parameters, which include the requested chromosome (REQCHROM), the requested start position (REQSTART), and the requested end position (REQEND) 1000. The logic initiates a resultant sequence buffer 1005 and sets a select_start_position variable equal to the requested start position minus 254 1010. The subtraction of 254 nucleotide positions assumes that the nucleotide sequences are stored in 255 segments, as in the example described above.

All records containing at least a portion of the desired sequence are retrieved. In particular, each segment is selected where the chromosome ID equals the requested chromosome (REQCHROM), the segment start is grater than or equal to the set select_start_position, and the segment start is less than the requested end position (REQEND) 1015. The result is a set of one or more selected segments.

Processing next determines whether more records exist from the set of selected segments 1020, and if “no”, processing is complete 1025. Assuming that more records exist, then processing determines whether the current record's start position is less than or equal to the requested start position (REQSTART) 1025. If “yes”, then an offset variable is defined, that is, OFFSETSTART=REQSTART−Current Record Start 1050. This can be seen in FIG. 11A, where the bolded sequence 1140 is to be retrieved from the segments of the table 1100, with the first segment to be retrieved beginning at position 511, and the requested start offset from that position. Thus, the offset start is calculated. Next, processing determines whether the end for that segment is greater than or equal to the requested end position (REQEND) 1055. Assuming “no”, then the current sequence is appended to the buffer from the offset start to the remainder of the segment 1060, and processing determines whether more records exist.

From inquiry 1055, if the current record end is greater than or equal to the requested end position, then processing sets a variable OFFSETEND equal to the OFFSETSTART+(REQEND−REQSTART) 1065. In the example of FIG. 11A, this results in the segment beginning with position 2041 being truncated to the requested ending position, as illustrated by the bolding. The current sequence is then appended to the resultant sequence buffer from the OFFSETSTART position to OFFSETEND position 1070.

From inquiry 1025, if the current record start is greater than or equal to the requested start position, then processing determines whether the current record end is greater than or equal to the requested record end 1030. If “no”, then the current sequence segment is appended to the resultant sequence buffer 1035, and processing determines whether more records exist. If “yes”, then the variable REMAININGLEN is set equal to REQEND−Current Record Start 1040, and the current sequence is appended to the buffer from index 0 to REMAININGLEN 1045.

As discussed above, the logic of FIG. 10 is configured to concatenate the proper portions of the retrieved sequence segments to generate the requested genomic sequence, as illustrated in FIGS. 11B & 11C. Advantageously, by employing a segmented sequence table and the processing of FIGS. 9 & 10, the seek time for a nucleotide sequence retrieval process becomes negligible, while still allowing for the benefits of storing the raw data in a database schema, such as discussed above.

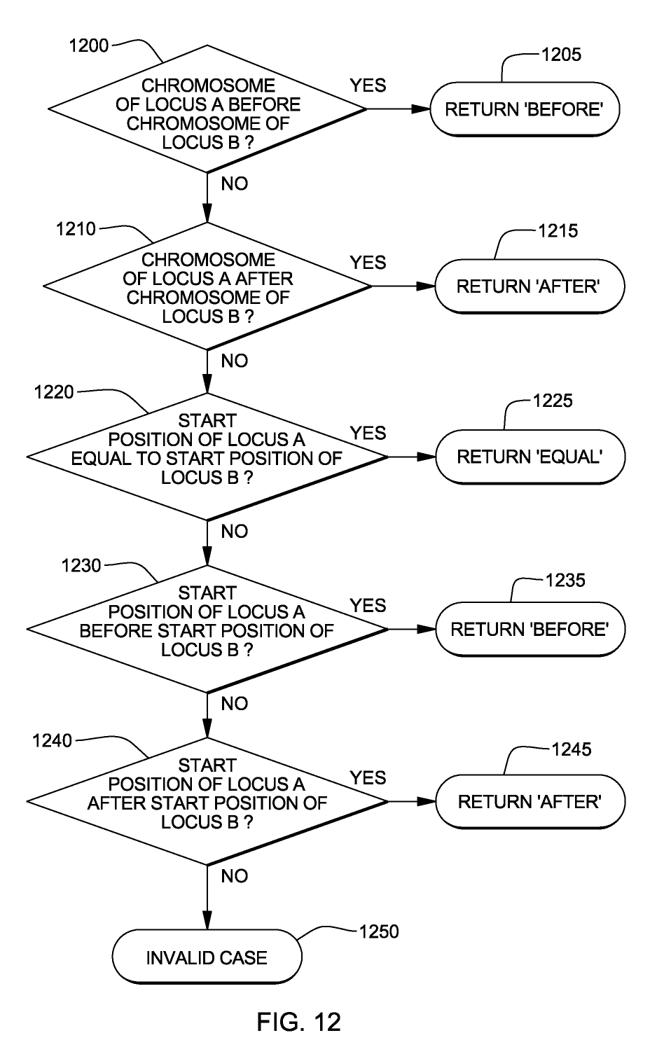

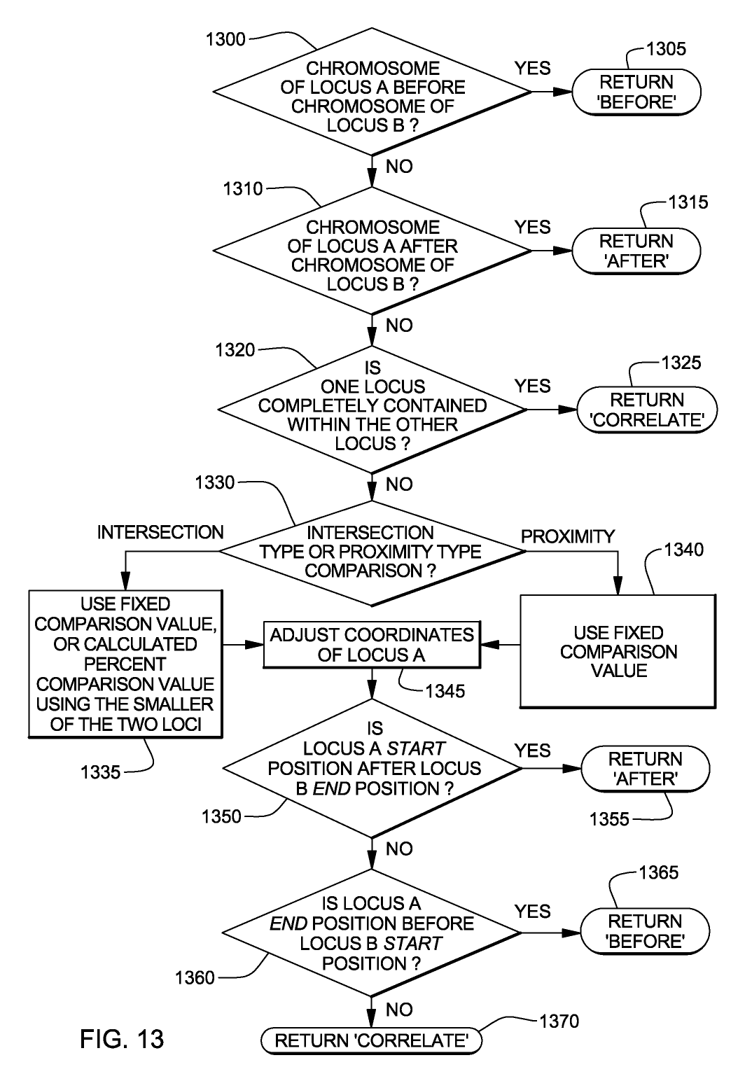

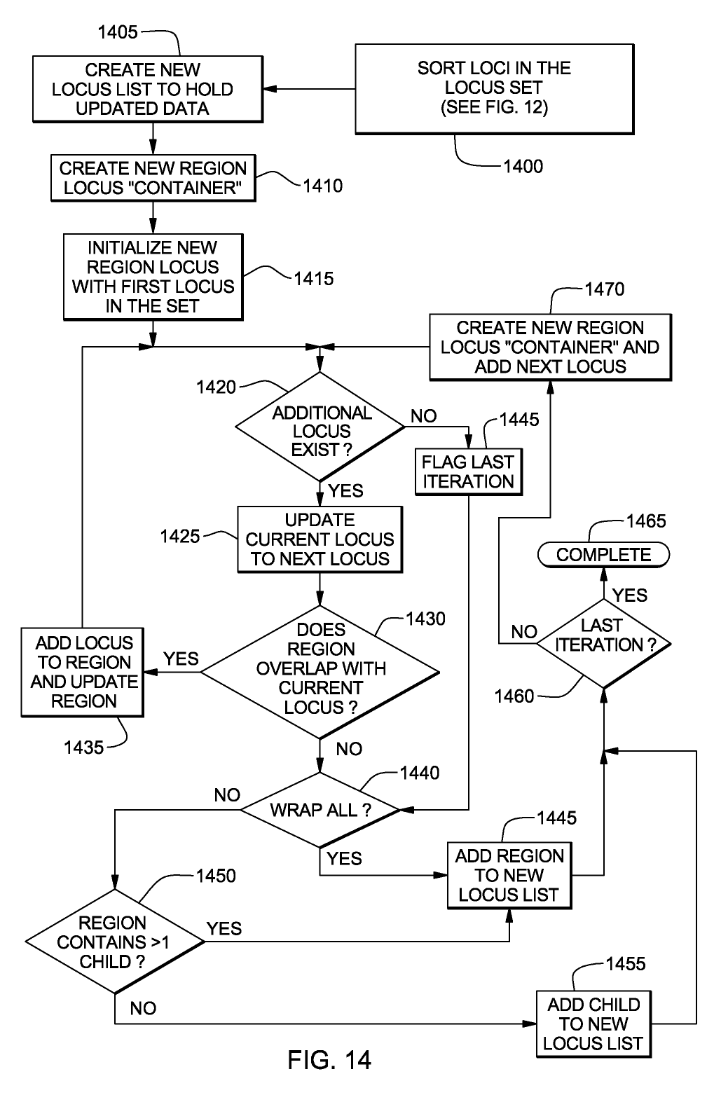

As noted above with reference to the data model discussion of FIG. 8, the locus object includes functionality or logic for facilitating sorting of locus objects, and comparison of locus objects for correlation. Examples of such locus sorting logic and locus comparison logic are illustrated in FIGS. 12 & 13, respectively.

Beginning with FIG. 12, locus object comparison for sorting begins with processing determining whether the chromosome of locus object A is before the chromosome of locus object B 1200. If “yes”, then a “Before” indication is returned 1205. If “no”, then processing determines whether the chromosome of locus object A is after the chromosome of locus object B 1210, and if “yes”, then an “After” indication is returned 1215.

Assuming that locus object A's chromosome is neither before or after locus object B's chromosome (meaning that the loci may be on the same chromosome), then processing determines whether the start position of locus object A is equal to the start position of locus object B 1220. If “yes”, then an “Equal” indication is returned 1225. Otherwise, processing determines whether the start position of locus object A is before the start position of locus object B 1230. If “yes”, then a “Before” indication is returned 1235. If “no”, then processing determines whether the start position of locus object A is after the start position of locus object B 1240. If “yes”, then an “After” indication is returned 1245. If “no”, an invalid case has been identified 1250, for example, representative of data error. In using the logic of FIG. 12, it can be seen that sorting is based on genomic coordinates (chromosome identifier and start position) of the two nucleotide loci being compared. One locus object is given to another locus object, and asked “how do you compare?” Answers include “Before”, “After”, or “Equal”. In the logic example of FIG. 12, it is considered that locus object A is being compared to locus object B. The comparison is contextual to the linear coordinate system to which both loci belong, i.e., the genomic coordinate system.

FIG. 13 depicts exemplary logic within each locus object for facilitating locus comparison for correlation (e.g., overlap). As explained in detail below, correlation analysis, in accordance with an aspect of the present description, may include selection of a comparison type and a comparison value to be used in performing the correlation analysis. Comparison type may be either intersection type or proximity type. Intersection type means that two loci being compared have at least partially intersecting nucleotide positions, while proximity type means that the loci being compared are within at least a defined number of nucleotide positions, that is, that the loci overlap or that the gap between loci is less than or equal to the defined number. The comparison value may either be a number (n) of nucleotide positions, wherein n≧1, or a percentage number (pn) or nucleotide positions, wherein pn≧0, which is employed in determining whether a first nucleotide locus (e.g., locus object A), and a second nucleotide locus (e.g., locus object B) correlate.

When intersection type is selected, correlation is defined by the first nucleotide locus and the second nucleotide sequence locus overlapping with at least the number (n) of nucleotide positions in common, or by the first nucleotide locus and the second nucleotide locus overlapping with at least the percent number (pn) of nucleotide positions in common relative to a smaller one of the first nucleotide locus and the second nucleotide locus. When proximity type is selected, correlation is defined by the first nucleotide locus and the second nucleotide locus being within at least the number (n) of nucleotide positions. Results of the correlation analysis can be output as an indication of “Before”, “After”, or “Correlate”.

By way of example, whether two loci correlate depends in one embodiment on what the user considers a valid correlation condition. For example, if two loci share a common region of only a single nucleotide, do they correlate?0 Or, does the shared region need to be at least 50 nucleotide positions? The user may instead prefer that a gap of some length be allowed between the two loci, while still maintaining a correlation condition. This flexibility of correlation definition is left to the user via selection of the comparison type and comparison value parameters. In addition, or as an alternative, default comparison type and comparison value parameters could be provided and utilized within the system, for example, in place of a user pre-selecting these parameters.

Note that in a further alternate implementation, comparison type may be defined as either fixed or percent, with fixed indicating a specific number of nucleotide positions that define the correlation criteria, whether intersection or proximity. For example, two loci might be required to share a region of at least 50 nucleotides, or the loci might be required to be within 1,000 nucleotide positions of each other, etc. Percent type, in this example, is a calculated percentage of the length which defines the intersect/proximity criteria. For example, two loci might correlate by at least 50%, with the percent number of nucleotide positions being calculated from the smaller number of the two loci. In this example, the comparison value may refer to either an integer value to accompany the fixed type, or a floating point value to accompany the percent type. In this implementation, it may be assumed that intersection type or proximity type may either be inherent in the options to be selected or fixed within the system for a particular application.

In FIG. 13, and the following discussion, it is assumed that comparison type refers to either intersection type or proximity type, while comparison value refers to either a number (n) of nucleotide positions, or a percent number (pn) of nucleotide positions. However, those skilled in the art should understand that the claims presented herewith are intended to encompass other implementations of these concepts, such as the above-noted fixed and percent type representations.

FIG. 13 again presents one embodiment of logic implemented within a locus object for facilitating comparison of two loci for correlation. Processing begins with determination of whether the chromosome of locus object A is before the chromosome of locus object B 1300. If “yes”, then a “Before” indication is returned 1305. If “no”, then processing determines whether the chromosome of locus object A is after the chromosome of locus object B 1310, and if “yes”, then an “After” indication is returned 1315. Otherwise, processing determines whether one locus object is completely contained within the other locus object 1320. If “yes”, then a “Correlate” indication is returned 1325. If “no”, then processing determines whether the user has selected intersection type or proximity type comparison 1330. If intersection type, then processing uses a user-selected fixed comparison value or a calculated percent comparison value, using the smaller of the two loci 1335. If proximity type, then the logic uses a user-selected fixed comparison value 1340.

In this embodiment, the coordinates of locus object A are then adjusted to facilitate the comparison process 1345. This adjustment may include increasing the start coordinate for the first nucleotide locus (i.e., locus object A) by the fixed number (n) of nucleotide positions or a number (x) of nucleotide positions, depending on the comparison type selected. In this example, and assuming intersection type selection, the number (x) is a required number derived from the percent number (pn) applied to the smaller of the two loci being compared. Additionally, the end coordinate for the first nucleotide locus is decreased by the same number (n) of nucleotide positions or number (x) of nucleotide positions to produce an adjusted start position and an adjusted end position for the first nucleotide locus. These adjusted positions are then used in the comparisons to follow. Specifically, processing determines whether the adjusted start position of locus object A is after the locus object B end position 1350. If “yes”, then an “After” indication is returned 1355. Otherwise, processing determines whether the adjusted end position of locus object A is before the start position of locus object B 1360. If “yes”, then a “Before” indication is returned 1365. If “no”, then a “Correlate” indication is returned 1370.

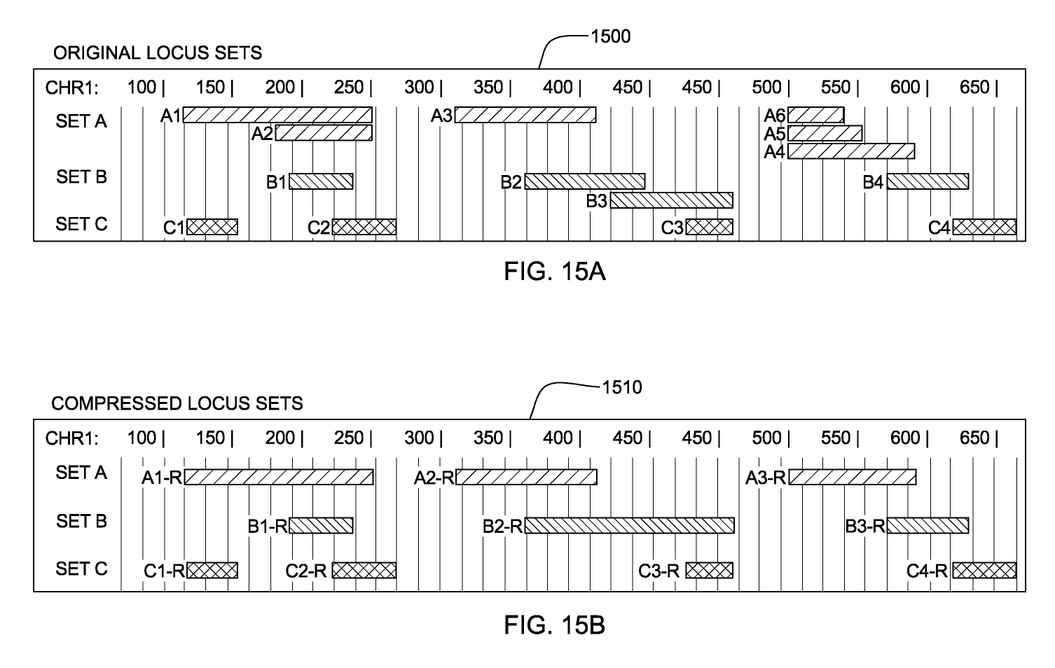

FIGS. 14, 15A & 15B illustrate one embodiment of the above-noted functionality within a locus set object for forming nucleotide regions within a locus set object. By way of example, this logic compresses or flattens the locus objects within the locus set object based on correlation. If two loci within a locus object set correlate, then the common region is added to a parent locus object. This parent locus object is refered to as a region, and acts as a container for the overlapping loci. This ensures that all loci directly contained within the locus set object are linear, and that the original data is maintained by the parent/child hierarchy.

More particularly, FIG. 14 depicts one example of logic within a locus set object for facilitating compression of nucleotide loci thereof into nucleotide regions to facilitate correlation analysis between different locus set objects. Processing begins with sorting the loci within the locus set object using, for example, the above-described processing of FIG. 12, which is resident within the locus objects within the locus set object 1400. Once sorted, a new locus list is initialized to hold the updated loci 1405 and a new region locus “container” is initialized 1410. A new region template is initialized with a first locus object (i.e., nucleotide locus) in the locus set object 1415, and processing determines whether more loci exist 1420. If “yes”, then the next locus object becomes the current locus object 1425, and processing determines whether the new region overlaps with the current nucleotide locus 1430. In one embodiment, “overlap” requires an intersection of one or more nucleotide positions between the loci being compared. Alternatively, the term “overlap” could be synonymous with correlation, as discussed above, in which case, the logic within the locus set objects may be configurable, or predefined such that overlap requires either intersection or proximity, and that the value of the intersection or proximity is predefined (and either fixed or based on a percent number). For example, two or more nucleotide loci may “overlap” or correlate for compression purposes into a single nucleotide region, with correlation defined as either intersection or proximity. For intersection type, each nucleotide loci pair being compared for compression either share at least a compression number (cn) of nucleotide positions in common, wherein cn≧1, or share a compression percent number (cpn) of nucleotide positions in common relative to a smaller one of the nucleotide loci pair undergoing compression analysis, wherein cpn≧0, and wherein for proximity, each nucleotide loci pair being considered for compression are within at least a compression range (cr) of nucleotide positions, wherein cr≧1. In one implementation, by default, the correlation type could be intersection type with an overlap of at least one nucleotide position. In such a case, the overlapping locus objects would, by default, be automatically compressed into a region.

Continuing with the processing of FIG. 14, if the answer to inquiry 1430 is “yes”, then the current locus is added to the new region and the new region is updated 1435. Thereafter, processing returns to consider whether an additional nucleotide locus exists within the data set 1420. If the current locus is the last locus in the data set, then a last iteration flag is set 1445. If the last iteration flag is set, or the current nucleotide locus does not overlap with the new region, processing inquires whether each new region locus is to be wrapped, that is, whether a single nucleotide locus (i.e., locus object) is to be maintained within a region container. This processing determines whether a region container is to be created for each single non-overlapping locus object, as well as for the overlapping locus objects 1440. If “yes”, then the new region is added to the new locus list 1445, and processing determines whether the last iteration flag has been set 1460. If “yes” again, then processing of the locus set object is complete 1465. Otherwise, a new region locus “container” is created and the next nucleotide locus is added to the new region container 1470, after which processing determines whether an additional locus exists within the locus set object 1420.

If a single nucleotide locus within the region container is not to be wrapped, then from inquiry 1440 processing inquires whether the region contains greater than one child locus 1450. If “no”, then the child locus is added to the new locus set (that is, is removed from the region container) 1455. Otherwise, the new region locus is added to the new locus list 1445.

FIGS. 15A & 15B illustrate a result of this processing. In FIG. 15A, three locus set objects (i.e., Set A, Set B & Set C) are illustrated 1500. These locus set objects may each contain loci which overlap within the locus set object. For example, reference loci A1 & A2 in Set A, and loci B2 & B3 in Set B, etc. Loci that overlap within each set are added to a region locus, using, for example, the processing of FIG. 14. Thus, locus A1 and locus A2 in Set A become Region A1-R, and locus B2 and B3 in Set B become Region B2-R in the illustration 1510 of FIG. 15B. Each region maintains information about the loci which it contains, but gives the locus set a linear data structure which can be used by the other logic presented herein. Further, the user can choose whether all loci are added to a parent container (i.e., a region locus), even if no overlaps are present, or if only overlapping loci are aggregated while leaving each unique nucleotide locus alone.

### Control Data Set Generation:

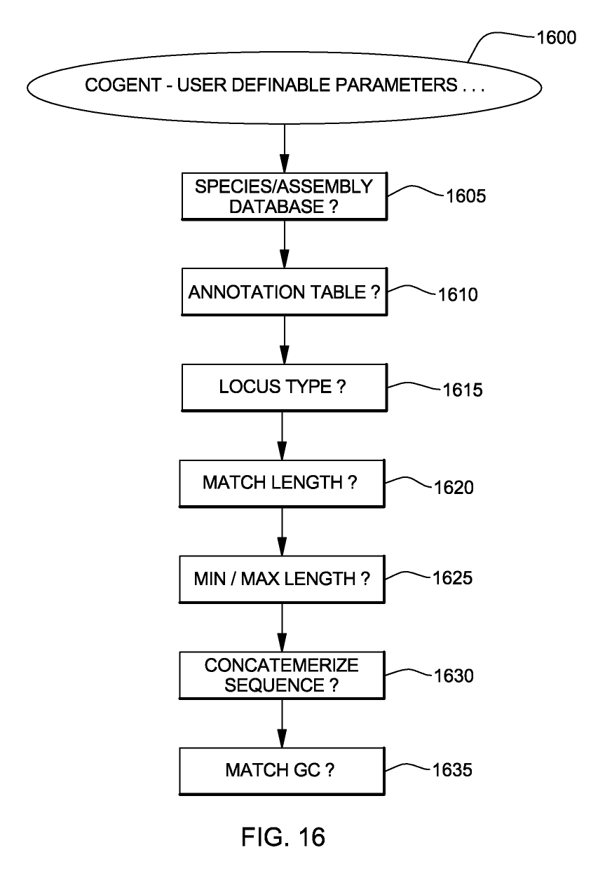

As noted above, control data set generation is also disclosed herein wherein a control generator tool/process creates matched data sets for facilitating informatic analysis. These matched data sets may include genomic loci and/or genomic sequences. The data is taken from a database of actual genomic data (including sequence and annotation data), as opposed to ad-hoc generation, sequence scrambling or the like. This produces biologically relevant and accurate results which allow for stronger controls. The controls are matched against a user-provided data set via a number of parameters, as illustrated in FIG. 16.

In FIG. 16 these user-definable parameters 1600 may include designation of a particular species/assembly database 1605, designation of a particular annotation table 1610, designation of a locus type 1615, designation of a match length 1620, selection of a minimum/maximum length 1625, designation of whether to concatemerize the sequence 1630 (where sequence parameters are applied to the nucleotide loci), and where sequence parameters are applied, designation of whether to match, for example, GC content 1635. The species, assembly and annotation designations refer to a particular database and table within the database to utilize (e.g., human_NCBI_B35-RefSeq) in the example of FIG. 5. The locus type designation allows the user to select a particular type of locus to retrieve from (e.g., gene, exon, UTR, etc.). The matching or min/max length selections allow a user to designate whether minimum/maximum or matching polynucleotide lengths are to be used. Essentially, the user is defining the stringency of the ultimate data selected. The min/max length designation would be an alternative to designating a requirement of matching length. By way of example, the respective loci within the control data set could match exactly the length of the corresponding loci within the experimental data set, or could be within minimum/maximum length settings, as defined by the user. The concatemerize sequence and match GC parameters refer specifically to genomic sequences and allow a user to designate whether to concatemerize selected genomic sequences to achieve a desired length, and whether to match GC content of the selected genomic sequences, that is, whether the occurrence of G and C within the genomic sequence is to be matched (in one example).

Note that the species/assembly database parameter, annotation table parameter and locus type parameter allow for user selection of the data population to be employed in generating the control data set. Each of these parameters is essentially a filter which qualifies where the control data is to be randomly selected from. The match length parameter, min/max length parameter, concatemerize sequence parameter and match GC parameter relate to attributes of the experimental data that are to be used to either accept or reject pieces of information being randomly retrieved to create the control data set. If desired, default settings for one or more of the parameters identified in FIG. 16 could be employed in one embodiment. However, multiple attributes of the experimental data set are to be employed in generating the control data set, thus resulting in a non-randomly generated control data set.

Control data generation logic, in accordance with one aspect of the invention disclosed herein, employs a database structure and access manager, as described above, which provide the user with a list of available species, assemblies, and annotations to choose from. The database manager, via the control generation tool, retrieves random data samples and filters this data based upon the user-defined parameters noted above. As described, these parameters can be contextual to the annotation (e.g., CDS only, 5′ UTRs, etc.), and they can be matched to the user's data set for greater control accuracy.

As an overview, a first data set is loaded into the control generation tool in the form of a locus set object. This represents the genomic loci or genomic sequences to be controlled. A matched control record is produced for each record in the data set, and each evaluated criteria is contextual to the current user record being examined. First, the user chooses which species/assembly database to be employed. Once selected, the user is presented with a list of annotation tables, and again a selection is made. Examples of annotation tables are: RefSeq, KnownGene, miRNAs, Transcription Factor Binding sites, Methylation, etc.

The user then sets parameters which will act as filters on the data. The first level filtering happens during data retrieval. A random sample is selected from the user-defined table, and only the specified loci are returned. The possible loci are contextual to the annotation table selected. For example, miRNAs would just have a single locus per record, while KnownGene could return whole gene regions, CDS, UTR, etc. This sample size is configurable, and is used to maintain a pool of data, thus minimizing database look-ups. The control generation tool then uses this pool of data and applies the second set of filtering criteria.

The logic branches, depending upon whether the user-requested sequences, or loci only. For the latter, the logic iterates over the loci in the pool and attempts to apply any length criteria (matching length, minimum length, maximum length, etc.). If the locus, or a subset, can meet the criteria, it is saved to the control set and the next user record is examined. Otherwise, it is discarded.

If the user-requested control is for a genomic sequence, then the actual nucleotide sequence is retrieved for the loci in the pool. The user can decide whether the control sequences should originate from a single concatemerized sequence. This avoids creating any “center selection” bias when randomly selecting regions from within a given locus. If this is the case, then an appropriate length sequence is selected with a random starting point, continuing across one or more sequences as needed to complete the length. If concatemerization is not required, then the logic iterates over the loci in the pool, and attempts to apply any length criteria (as described above). Once an appropriate length sequence is found, it is checked for matching GC content. GC content can be set to match a given percentage threshold from ±100% (GC does not need to be matched) to ±5% (for example). If the locus matches required GC content, it is saved to the control set, and the next user record is examined. Otherwise, it is discarded.

Once all records in the user-defined table set have a matched control, processing exits and the control set is output, for example, to the user.

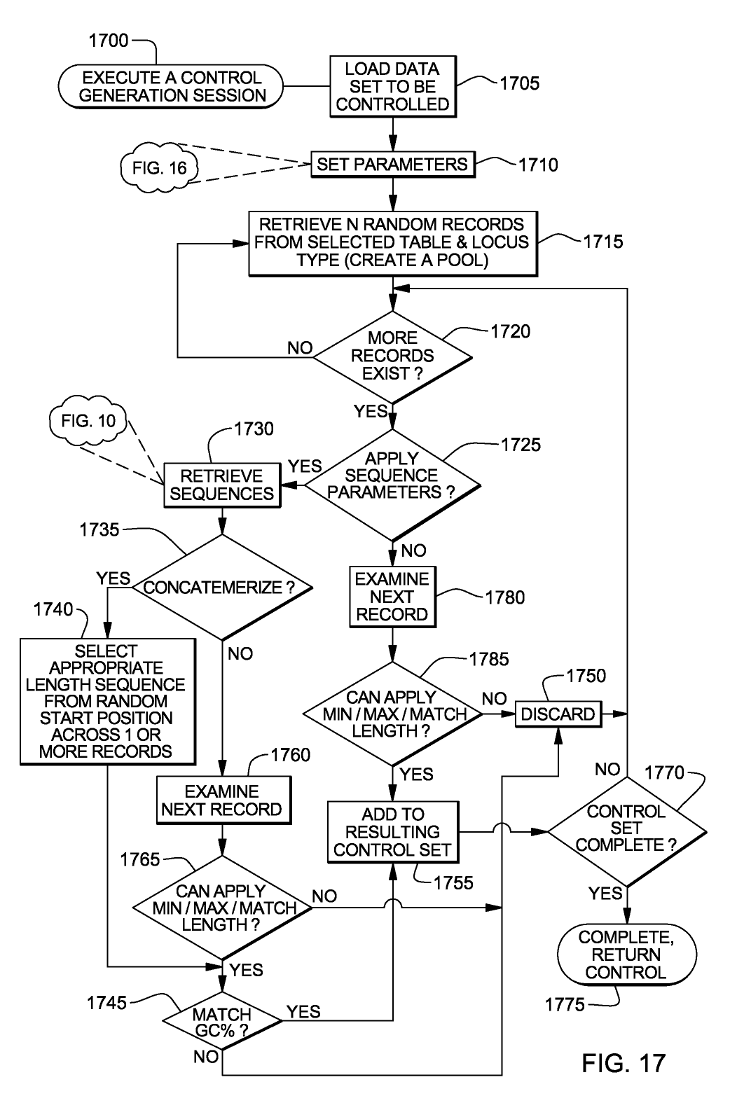

FIG. 17 depicts one detailed example of this logic. A control generation session or instance is created 1700, and the data set to be controlled is loaded 1705 (i.e., the data set for which a control data set is to be generated is loaded). Parameters, such as those described above in connection with FIG. 16 are set, for example, by a user 1710. N random records are retrieved from the selected table and locus type to create a pool of data 1715. This use of a pool of records from the database minimizes database retrievals. Processing initially determines whether more records exist within the pool 1720. If “no”, then N random records are again retrieved from the selected table and locus type to create another pool. If more records exist, then processing determines whether sequence parameters are to be applied 1725. If “yes”, then the appropriate sequences are retrieved 1730, using, for example, the processing of FIG. 10. Processing next determines whether to concatemerize the sequences 1735. If “yes”, then the records are concatemerized and the appropriate length sequence is selected from a random start position across one or more records 1755. By default, this selection results in the exact length desired for the particular control. Processing then determines whether the GC content in the selected sequence length matches the set parameter 1760. If “no”, then the sequence is discarded 1750. Otherwise, the sequence is added to the resulting control set 1755.

If concatemerize sequence is not employed, then a next record is examined 1760, and processing determines whether a min/max/match length designation can be applied to the record 1765. If “no”, then the record is discarded 1750. Otherwise, the record is examined for a matching GC content 1745, as described above.

After adding a loci or sequence length to the control set, processing determines whether the control set is complete 1770. If “yes”, then the control set is returned to the user or system, for example, for use in correlation analysis, as described herein. If the control set is not complete, then processing determines whether more records exist within the pool 1720. If processing is not to apply sequence parameters to the pool of records, then processing examines the next record 1780 and determines whether the record meets the minimum/maximum/match length designation set by the user 1785. If “no”, then the record is discarded 1750, and if “yes”, the record is added to the control data set. The result is a control data set wherein loci within the data set correlate to loci within the initially-loaded data set to be controlled. This intelligent selection of loci results in a control data set which is matched closely to the user-provided data set and thus produces more biologically relevant and accurate results when using the control data set, for example, for comparison purposes in correlation analysis with a third data set.

### Correlation Analysis:

The correlation analysis tool of the system performs correlation analysis for sets of genomic loci. It performs comparisons among coordinate-based data in a high throughput manner, identifying shared or common regions. The tool allows for any number of sets of loci to be compared, with each set containing any number of loci, which may overlap within a set. A variable number of nucleotides can be defined for each minimum required correlation, or maximum allowed gap between loci. This minimum overlap or maximum gap can be set either as a fixed number, or a percentage, as described above. Also, any set can be defined as a negative set, meaning it should not be in common with the others. Further, a “bridging” criterion is allowed, where a locus can span two other loci and bridge the intervening region. The correlation analysis tool is rooted in a simple set intersection analysis. However, the data and compare conditions hold additional complexity. Each group of loci is a set which can intersect with other sets. But each set member (i.e., each nucleotide locus) is not a discrete unit which can be defined as a member of multiple sets. In fact, each locus is itself a set (of nucleotides) and the nucleotides act as the discrete unit of comparison. Thus, the requirement becomes an analysis of sets of sets.

There are caveats within the conditional comparisons as well. For instance, multiple loci within the same set are able to intersect with each other (e.g., isoforms of a gene). Also, when comparing loci, the determination of a true/false intersecting condition is variable, given the user-defined parameters. This means that loci can share any number of nucleotides, or even none at all (allowing for a proximity analysis), and still be considered a true condition. Further, a bridging criteria can be considered, which forces a simultaneous comparison among elements of three or more sets, allowing for more complex truth conditions. To maximize efficiency, the correlation analysis tool applies an ordered set and sweep concept to move through the data. (The ordered set and sweep is conceptually similar to the Bentley-Ottoman algorithm for finding the set of intersection points for a collection of line segments in two-dimensional space.) The correlation analysis tool orders loci within each input set based on their genomic coordinates. This allows the tool to organize each data set in a virtual linear model, and then “sweep” across them, minimizing the number of comparative permutations that must be generated. Due to the possibility of intersecting loci within a single set, there are a minimum number of iterative permutations that must be computed. However, by utilizing the ordered nature of the data and hierarchical data structures, these permutations are isolated to many small scopes, and the resource requirement is minimal.

In LCA (locus correlation analysis) the loci are addressed in a linear order within their context, and directionality is implicit within the coordinates. It doesn't matter whether the biological directionality of the loci is 5′→3′, p→q. etc; and LCA does not need to make any assumptions. However for reference purposes, the end of the context with the lowest number coordinates is referred to as the “low end”, and the end of the context with the highest number coordinates is referred to as the “high end”. Thus the locus closest to the low end is referred to as the “low-end locus”. The next locus in order is the “next low-end locus”, etc. Input data sets can be defined in two ways: they “should intersect” or they “should not intersect”. Sets that should intersect are referred to herein as “positive sets”, and sets that should not intersect are referred to herein as “negative sets”.

### Assumptions, Data Types and Configuration:

1. Input data: LCA accepts data in the form of locus set objects (as defined above in Database Schema and Data Model).
2. Assumptions: LCA assumes that the input data shares the same genome context-such as species, build number, etc., as well as the same coordinate system. Also, LCA assumes that in each locus set, the loci of interest are those directly referenced by the locus set. If any locus objects within the locus set contain a hierarchy (they have ‘children’ loci), the hierarchy is not recursed and child loci are ignored.
3. Bridging: Bridging is the condition in which 3 or more loci are being compared, and all loci only need to intersect with one other locus. For example: assume loci A, B, and C. A & B do not intersect, however if A & C do intersect and B & C do intersect, then C bridges A & B, and all three are considered to intersect or correlate.
4. Comparison type & comparison value: These parameters represent what the user defines as a true condition each time 2 loci are being compared. They are the same parameters as defined above and indeed LCA utilizes this functionality directly as it proceeds through the analysis.
5. Non-Intersecting/Not in Common: The non-intersecting criteria allows for the negative condition to exist. Any data set that is loaded into LCA can be defined as not in common (negative), and should not intersect with the other data sets. For example, one could load Set 1 (experimental results) to be intersecting with Set 2 (phylogenetically conserved regions) and non-interesting with Set 3 (all genes). Thus the result would be conserved experimental loci that are intergenic.
6. Output: LCA produces 3 types of results:
   a. A subset of each original set, representing the loci which resulted in a positive condition.
   b. A set of regions, representing the aggregated loci which intersected with each other. These regions provide information about the union and intersection, as well as the original data points.
   c. A matrix representing the specific, unique groups of loci which intersected across all data sets.

Each locus set given to LCA is prepared before the comparison processing begins. First the locus sets are copied, in order to preserve the integrity of the original sets. Then they are ordered, as described above. Lastly, the locus sets are compressed, again as described above. This is done because the sweeping process could fault in certain instances when the data sets are not linear (i.e., multiple loci overlap within the same set). For the compression process, the “Wrap All” parameter is used to tell the locus set to place all locus objects into a region container, as described above. This would give the LCA logic a consistent data structure to work with.

The logic maintains a reference to one region from each set. The referenced regions are determined in an iterative fashion by virtually sweeping along the genomic data and finding which set has the next low-end region. Once it is found, that set's reference is changed to the newly discovered region, the referenced regions from the sets are evaluated for intersection, and the sweep continues.

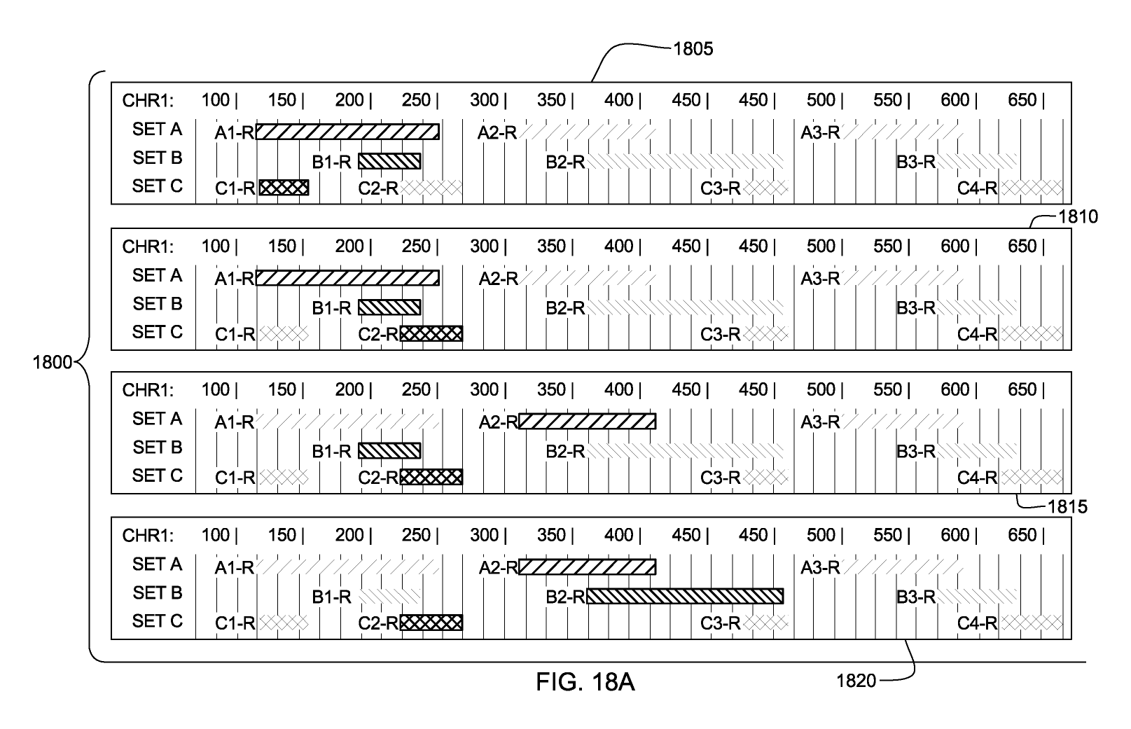

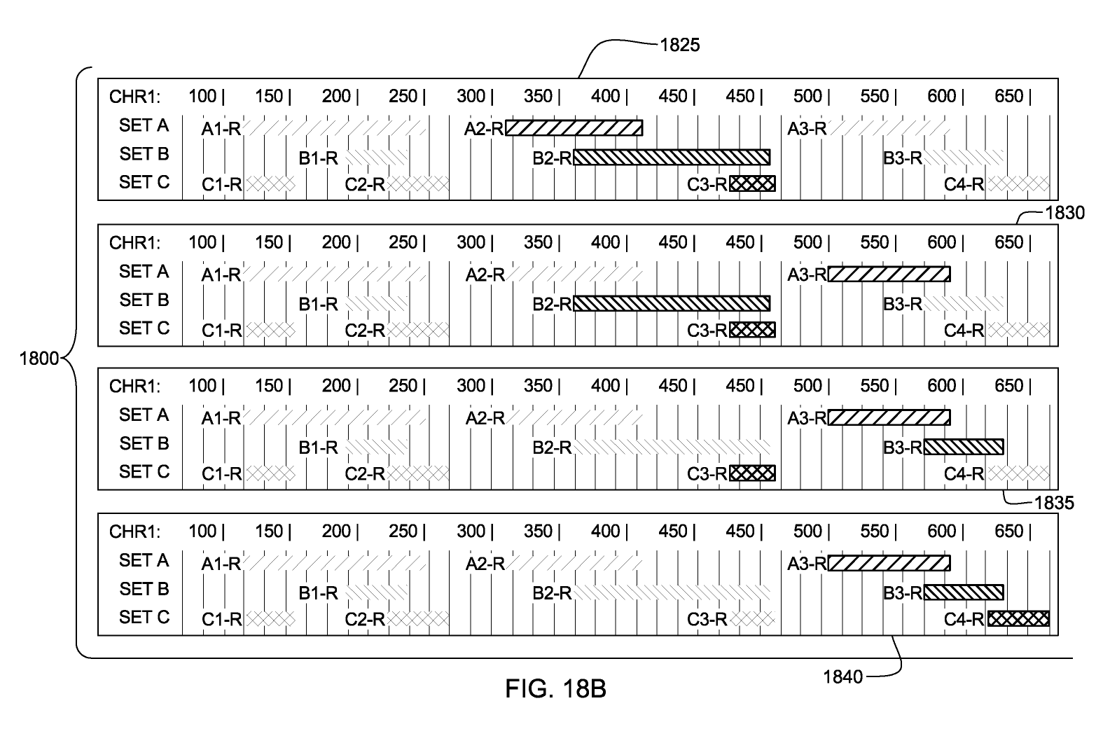

For example, in FIGS. 18A & 18B, there are 3 sets (Set A, Set B & Set C) of positive regions represented 1800. The first regions to be referenced and compared from the sets are A1-R, B1-R, and C1-R 1805. After the comparison is made, each set is tested for existence of another region. Of the sets that do have another region (in this case they all do: A2-R, B2-R, and C2-R) those regions are examined. C2-R is selected, and the comparison is made among A1-R, B1-R and C2-R 1810. Next, Set A's current reference is changed to region A2-R, and the comparison is made among A2-R, B1-R and C1-R 1815. This procedure continues until all regions have been exhausted 1820-1840.

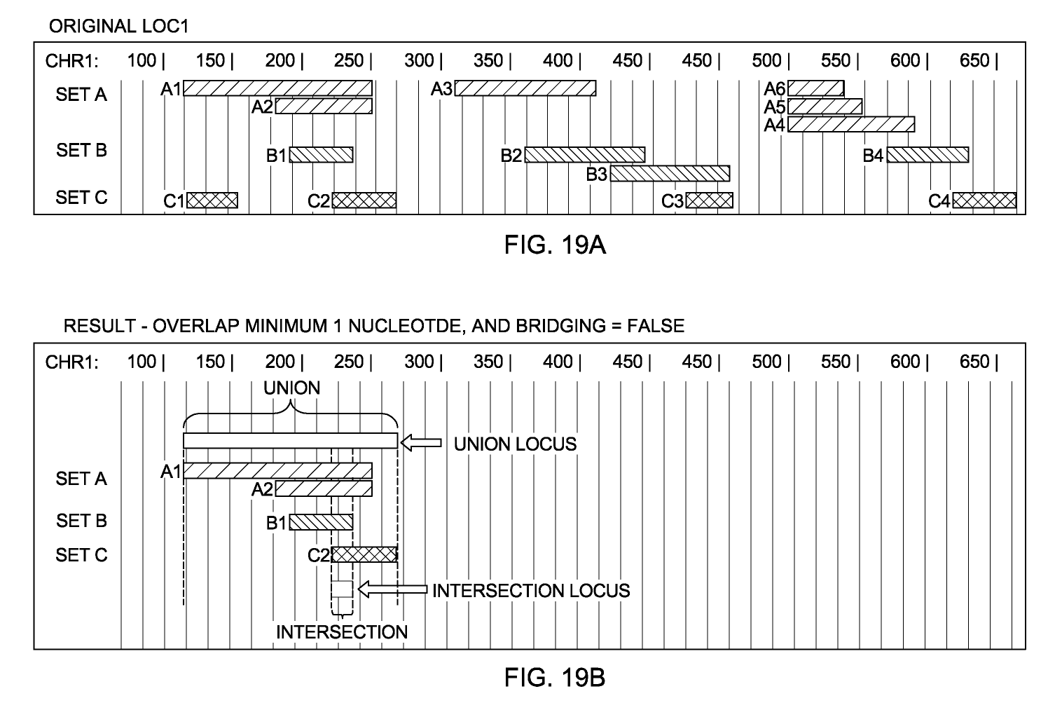

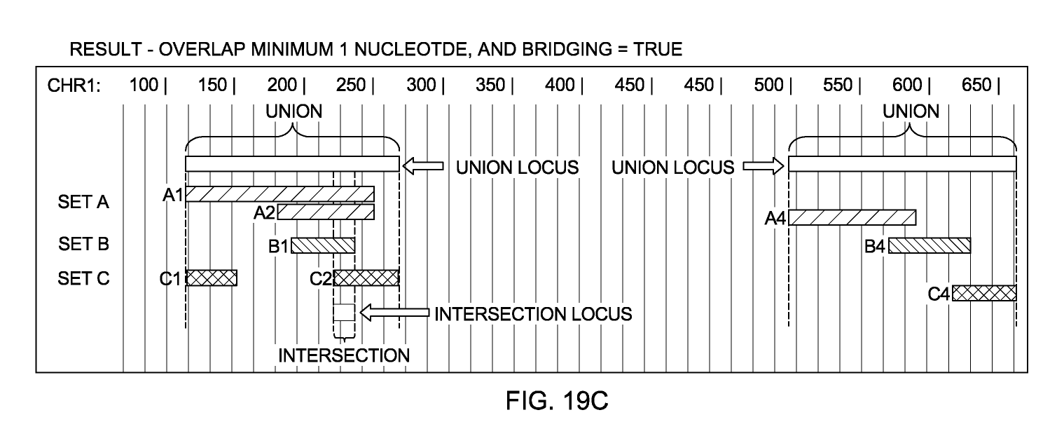

Each time regions are evaluated for intersection, the logic accounts for the user defined parameters of minimum overlap or maximum gap, and bridging. As stated previously, bridging allows for a true condition (i.e., a common region) among 3 or more loci. For example, in FIG. 19A, when comparing Sets A1, B1, and C1, it is seen that the sets do not share a common region and the condition is considered negative without bridging, as shown in FIG. 19B. However if bridging is allowed, then locus A1 bridges B1 and C1, and the condition is considered positive, with the result shown in FIG. 19C. The same phenomena appears when the comparison is made among loci A4, B4 and C4. The comparison of these loci results in a negative condition without bridging, and a positive condition with bridging.

Each time referenced regions are determined to be positive for intersection, the logic branches. When this occurs, all permutations for the individual loci contained within the regions are examined. Each permutation of loci is evaluated for intersection, using the same criteria as the region comparisons. If a positive condition is found, then the negative data set condition is checked.

The negative locus sets are treated similarly to the positive data sets, except they are aggregated into a single locus set to reduce the conditional load. The negative locus set maintains a reference, which keep track of the current scope (genomic coordinates) of the positive regions. This allows for ‘checks’ against negative regions to be held to a minimum, since only negative regions within the current scope need to be checked. When positive intersecting regions are found, references to the negative regions are evaluated. If the currently referenced negative region is “before” the first positive region, then the reference is moved up to the next negative region. This process repeats until the current negative region is no longer before the first positive region (and thus is no longer out of scope). After the negative region reference has been updated, the permutations of loci within the positive regions are checked. When an intersection of loci is found, processing compares these loci to the negative regions. The comparison starts at the currently referenced negative region (which is now in scope), and continues to compare against consecutive negative regions, but only until the negative regions are “after” the last positive region (and thus out of scope).

As the iteration proceeds, each group of loci which have passed the criteria are processed as positive results. This includes:
1. Flagging all positive locus objects from each locus set with a LCA-specific attribute. This allows LCA to quickly aggregate and return the subset of loci from each original locus set which passed the user's criteria. The return value is simply another locus set object.
2. Assigning each positive group of loci to another data structure called a locus nexus. This functional matrix represents each specific locus that intersects with each other specific locus. This tells the user what exactly from Set A intersects with what exactly from Set B, etc., as illustrated by the following table using data from FIG. 19C:

| Set A | Set B | Set C |
|---|---|---|
| A1 | B1 | C1 |
| A1 | B1 | C2 |
| A2 | B1 | C2 |
| A4 | B4 | C4 |

3. Assigning each positive locus to an aggregate region. These regions are locus objects which act as containers for positive loci. They perform 3 functions. They represent the largest total area occupied by all loci in the region—the Union. They hold all the original locus objects which make up the region, tracking their annotation and the locus set they came from. Lastly, they hold additional locus objects representing the region(s) of intersection. See FIG. 19C.

Any of the above result types can be requested from the LCA logic after a single iteration of the processing. Each presents the results in a different manner, and which type the user chooses depends on the question(s) being asked.

Those skilled in the art should note that the displays of FIGS. 19B & 19C are presented by way of example only. Further, when these representations are employed, a user could interactively click on any one of the displayed locus to obtain the relevant genomic data, for example, particular genomic sequence. In this respect, the displays of FIGS. 19B & 19C build upon prior state of the art with respect to visualization of genomic data. In addition, or alternatively, the concepts presented herein may be employed in a high throughput implementation where, for example, a user might be presented with a list or table of genomic data which corresponds to intersecting nucleotide positions of two or more nucleotide loci. The timing and format of the output provided can be selected for a particular implementation.

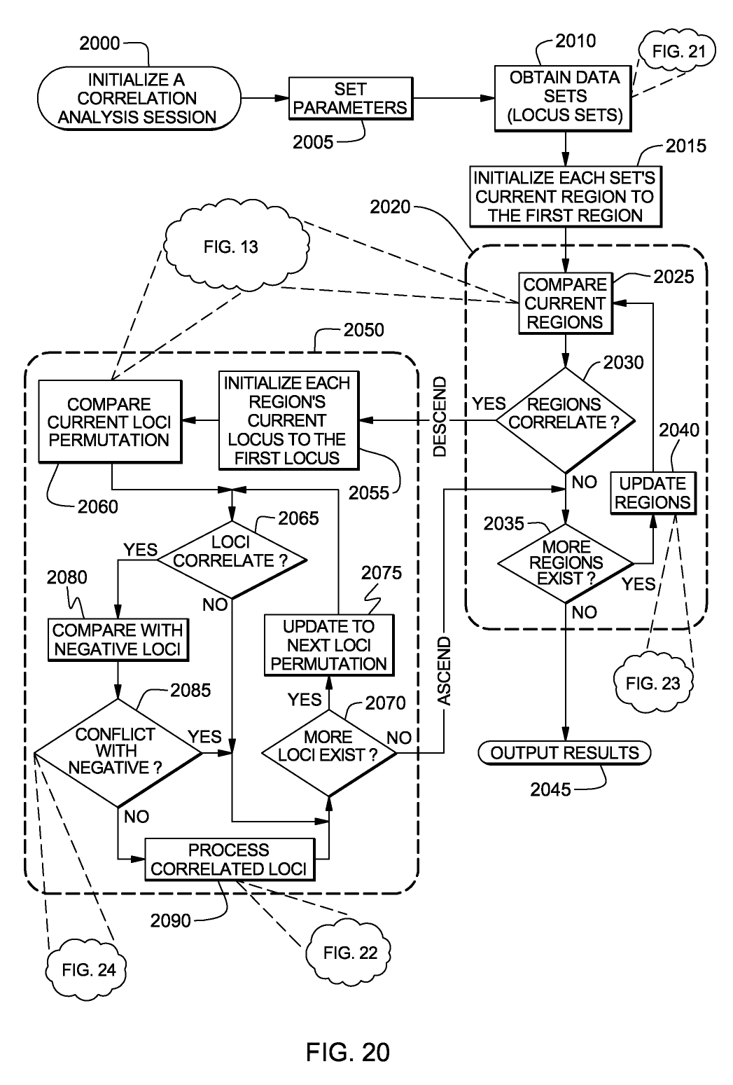

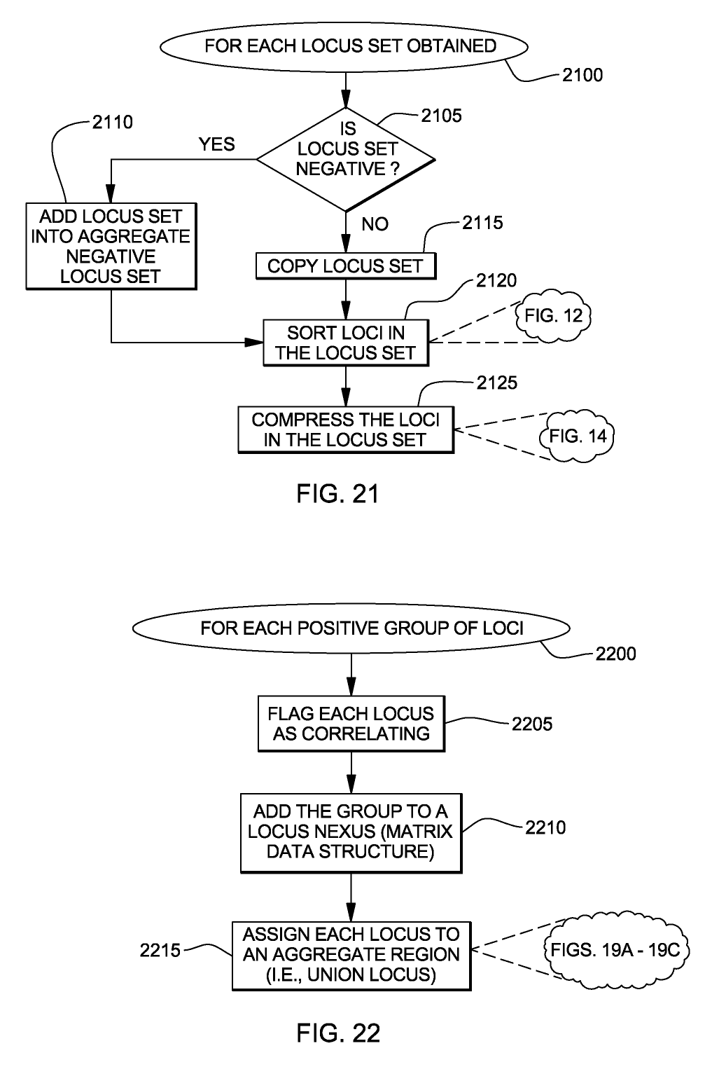

FIG. 20 depicts one example of the above-described logic for performing correlation analysis between loci of two or more locus sets. A correlation analysis session is initialized 2000 and parameters are set 2005, including, for example, one or more of the above-described bridging, comparison type and comparison value, non-intersecting/not-in-common, and output parameters. The data sets are obtained 2010, as set forth, for example, in FIG. 21.

Referring to FIG. 21, for each locus set object obtained, processing determines whether the locus set is user-defined as negative 2105. If “yes”, then the locus set is added to an aggregate negative locus set 2110. The aggregate negative locus set is a single locus set which aggregates all locus sets defined by the user as negative. If the locus set is not defined as negative, then the locus set is copied for manipulation, thereby retaining the original information. Loci within the locus set are sorted 2120, as described above in connection with FIG. 12, and then compressed into regions, as discussed above in connection with FIG. 14.

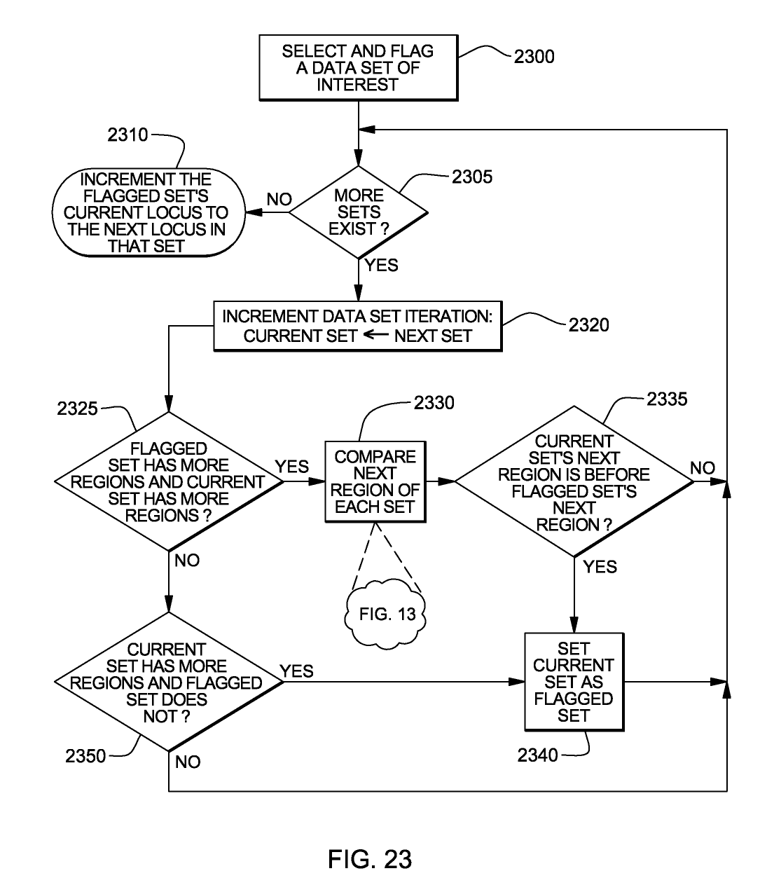

Continuing with the logic of FIG. 20, processing next initializes each set's current region to the first region at one end of the genomic coordinate system 2015. Next, processing 2020 is performed for positive overlapping regions within the data sets. This processing includes comparing the current regions 2025 and determining whether the regions correlate 2030. Correlation again can be user-defined, as described above, employing comparison type and comparison value parameters. If “no”, then processing determines whether more regions exist within the data sets 2035. If again “no”, then the results are output 2045. Otherwise, the set of regions being compared is updated 2040 as described above in connection with FIGS. 18A & 18B. One embodiment of this update logic is presented in FIG. 23.

Referring to FIG. 23, a data set of interest is selected and flagged 2300, and processing determines whether more data sets exist 2305. If “no”, then the flagged set's current locus is incremented to the next locus in that set 2310. If “yes”, then the data set iteration is incremented to the next data set 2320, and processing determines whether the flagged data set has more regions and the current set has more regions 2325. If “yes”, then the next region of each data set is compared 2330 using, for example, the processing of FIG. 13 described above. Processing then determines whether the current set's next region is before the flagged set's next region 2335. If “no”, then processing determines whether more sets exist 2305. If “yes”, then the current set becomes the flagged set 2340. Returning to inquiry 2325, if the flagged set and the current set do not each have more regions, processing determines whether the current set has more regions and the flagged set does not 2350. If “yes”, then the current set becomes the flagged set 2340. Otherwise, processing returns to determine whether more sets exist 2305.

Returning to FIG. 20, if regions correlate 2030, processing descends into the correlated regions to evaluate the loci thereof using logic 2050. Specifically, each region's current locus is set to the first locus therein 2055 and processing compares the current loci permutation 2060 to determine whether those loci correlate 2065. If “no”, then processing determines whether more loci exist within the regions 2070, and if “yes”, the loci are updated to the next permutation 2075, and processing considers whether the next permutation of loci correlate 2065.

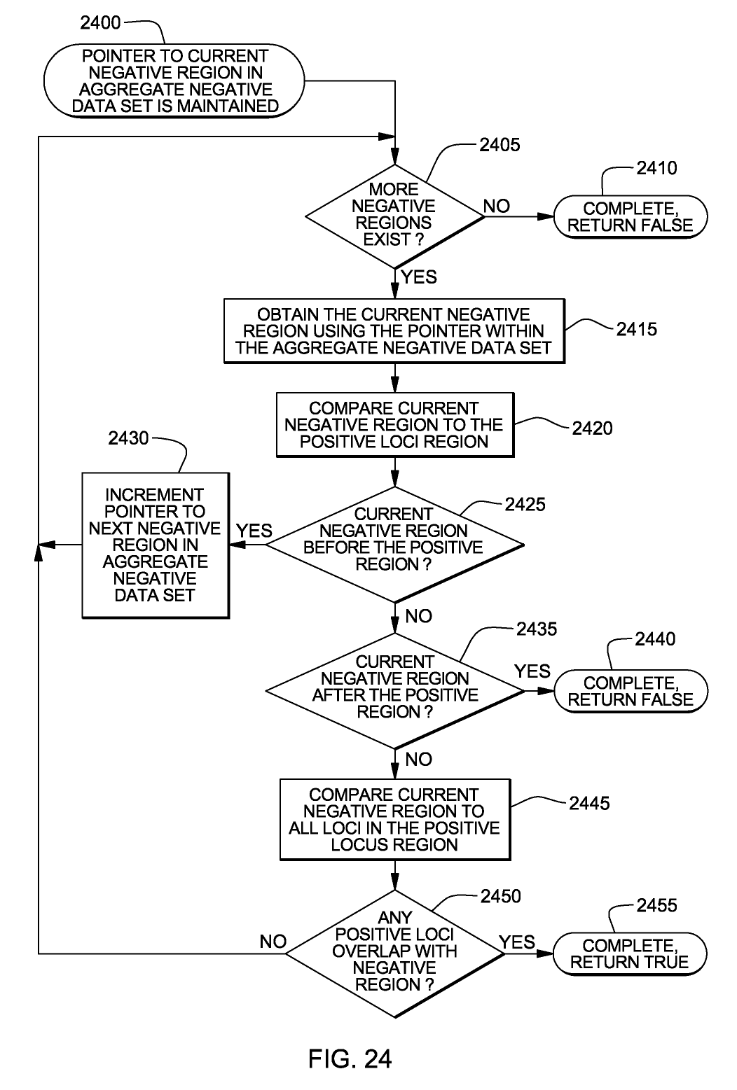

If the loci correlate, then from inquiry 2065, processing compares the correlated loci with the aggregate negative data set, or more particularly, with the negative loci therein 2080 and determines whether the correlated positive loci conflict with one or more negative loci within the aggregate negative data set 2085 using, for example, the logic of FIG. 24.

Referring to FIG. 24, from a pointer maintained to the current negative region in the aggregate negative data set 2400, processing determines whether more negative regions exist 2405. If “no”, then processing is complete and a false designation is returned, meaning that there is no conflict with a negative region 2410. If “yes”, then the current negative region is obtained using the maintained pointer 2415. This current negative region is compared to the positive correlated loci region 2420. Processing determines whether the current negative region is before the positive correlated region 2425. If “yes”, then the negative region pointer is incremented 2430, and processing returns to determine whether more negative regions exist 2405.

If the current negative region is not before the positive region, then processing determines whether the current negative region is after the positive region 2435. If “yes”, then processing is complete, and a false indication is returned, meaning that there is no overlap with a negative region of the aggregate negative data set 2440.

If the current negative region is not before or after the positive correlated region, processing compares the current negative region to all loci in the positive correlated region 2445, and determines whether any positive loci overlap with the current negative region 2450. If “yes”, then a true indication is returned, meaning that the correlated loci are not to be processed 2455. If “no”, then processing loops back to determine whether more negative regions exist within the aggregate negative data set 2405.

Returning to FIG. 20, and as noted above, if the correlated loci conflict with one or more negative regions of the aggregate negative data set, then processing determines whether more loci exist 2070. If there is no conflict with a negative region, then the correlated loci are processed, as described in FIG. 22, after which processing again determines whether more loci exist 2070. If “no”, then processing returns to region level processing to determine whether more regions exist 2035.

FIG. 22 depicts one example of processing which may be performed on the correlated loci. For each positive group of correlated loci 2200, each locus therein is flagged as correlating 2205, and the group is added to a locus nexus 2210, which is a matrix data structure such as discussed above in connection with FIGS. 19A-19C. Each locus is assigned to an aggregate region of the data structure 2215, that is, it becomes part of the associated union locus. As illustrated in FIGS. 19B & 19C and discussed above, each defined data structure, in addition to the union locus, includes the original correlated nucleotide loci within the group, and an intersection locus identifying nucleotide positions overlapping between the correlating nucleotide loci of the data sets.

FIG. 25 depicts one example of a display of output results provided to a user employing a system such as described herein above. A user interface 2500 includes a content or data view area 2510 including a flow diagram of the processing, with a representation of user-provided data sets 2520, a representation of the use of the control generator tool 2525 to generate a control data set 2530, and a representation of performing correlation analysis 2535 on, for example, the control data set compared with an existing mapped data set 2540, such as RefSeq Genes, with the result of the correlation analysis also being provided 2550. This flow diagram allows a user to interactively examine the data sets, parameters employed in one or more stages thereof, and the results of the various processing selected. This interactivity is indicated by pop-up windows 2555 wherein additional information on one or more displayed data sets or process steps of the logic may be provided to the user. The various items in the flow diagram may be represented using shapes, colors, or both. Relationships may be shown via connecting arrows. In addition to interacting with the individual elements to show additional information, the user may download data sets from the flow diagram. Additionally, the flow diagram can be converted to an image file for documentation purposes.

The detailed description presented above is discussed in terms of program procedures executed on a computer, a network or a cluster of computers. These procedural descriptions and representations are used by those skilled in the art to most effectively convey the substance of their work to others skilled in the art. They may be implemented in hardware or software, or a combination of the two.

A procedure is here, and generally, conceived to be a sequence of steps leading to a desired result. These steps are those requiring physical manipulations of physical quantities. Usually, though not necessarily, these quantities take the form of electrical or magnetic signals capable of being stored, transferred, combined, compared, and otherwise manipulated. It proves convenient at times, principally for reasons of common usage, to refer to these signals as bits, values, elements, symbols, characters, terms, numbers, objects, attributes or the like. It should be noted, however, that all of these and similar terms are to be associated with the appropriate physical quantities and are merely convenient labels applied to these quantities.

Further, the manipulations performed are often referred to in terms, such as adding or comparing, which are commonly associated with mental operations performed by a human operator. No such capability of a human operator is necessary, or desirable in most cases, in any of the operations described herein which form part of the present invention; the operations are automatic machine operations. Useful machines for performing the operations of the present invention include general purpose digital computers or similar devices.

Each step of the methods described may be executed on any general computer, such as a server, mainframe computer, personal computer or the like and pursuant to one or more, or a part of one or more, program modules or objects generated from any programming language, such as C++, Java, Fortran or the like. And still further, each step, or a file or object or the like implementing each step, may be executed by special purpose hardware or a circuit module designed for that purpose.

Aspects of the invention are preferably implemented in a high level procedural or object-oriented programming language to communicate with a computer. However, the inventive aspects can be implemented in assembly or machine language, if desired. In any case, the language may be a compiled or interpreted language.

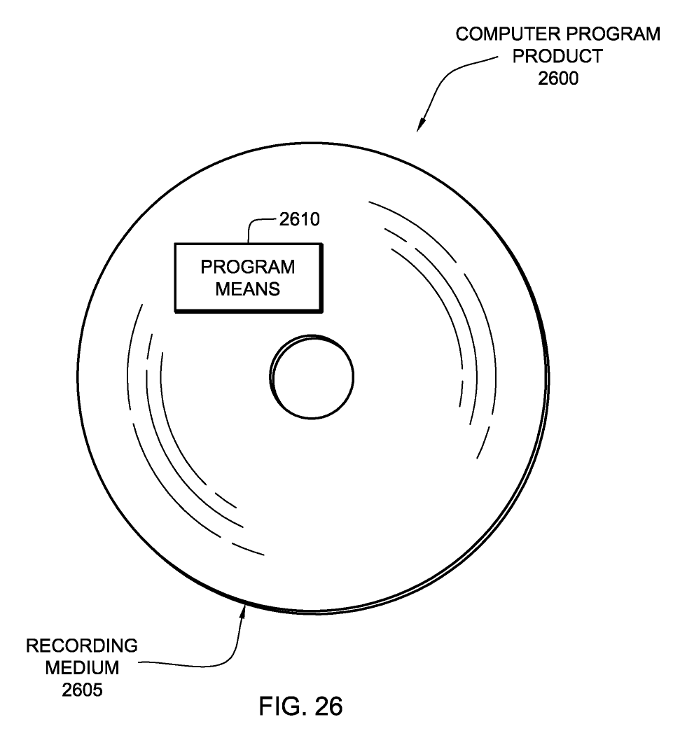

The invention may be implemented as a mechanism or a computer program product comprising a recording medium such as illustrated in FIG. 26. A computer program product 2600 includes, for instance, one or more computer-usable media 2605 to store computer readable program code means or logic 2610 thereon to provide and facilitate one or more aspects of the present invention. Such a mechanism or computer program product may include, but is not limited to CD-ROMs, diskettes, tapes, hard drives, computer RAM or ROM and/or the electronic, magnetic, optical, biological or other similar embodiment of the program. Indeed, the mechanism or computer program product may include any solid or fluid transmission medium, magnetic or optical, or the like, for storing or transmitting signals readable by a machine for controlling the operation of a general or special purpose programmable computer according to the methods of the invention and/or to structural components in accordance with a system of the invention.

The invention may also be implemented in a system. A system may comprise a computer that includes a processor and a memory device and optionally, a storage device, an output device such as a video display and/or an input device such as a keyboard or computer mouse. Moreover, a system may comprise an interconnected network of computers. Computers may equally be in stand-alone form (such as the traditional desktop personal computer) or integrated into another environment (such as a partially clustered computing environment). The system may be specially constructed for the required purposes to perform, for example, the method steps of the invention or it may comprise one or more general purpose computers as selectively activated or reconfigured by a computer program in accordance with the teachings herein stored in the computer(s). The procedures presented herein are not inherently related to a particular computing environment. The required structure for a variety of these systems will appear from the description given.

Further, one or more aspects of the present invention can be provided, offered, deployed, managed, serviced, etc., by a service provider. For instance, the service provider can create, maintain, support, etc., computer code, a relational database array, and/or a computer infrastructure that performs one or more aspects of the present invention for one or more customers. In return, the service provider can receive payment from the customer under a subscription and/or fee arrangement, as examples. Additionally, or alternatively, the service provider can receive payment from the sale of advertising content to one or more third parties.

In one aspect of the present invention, an application can be deployed for performing one or more aspects of the invention. As one example, the deploying of the application comprises adapting computer infrastructure operable to perform one or more aspects of the present invention.

As a further aspect of the present invention, a computing infrastructure can be deployed comprising integrating computer-readable program code into a computing system, in which the code, in combination with the computing system, is capable of performing one or more aspects of the present invention.

As yet a further aspect of the present invention, a process for integrating computer infrastructure, comprising integrating computer-readable program code into a computer system may be provided. The computer system comprises a computer-usable medium, in which the computer-usable medium comprises one or more aspects of the present invention. The code, in combination with the computer system, is capable of performing one or more aspects of the present invention.

The capabilities of one or more aspects of the present invention can be implemented in software, firmware, hardware or some combination thereof. At least one program storage device readable by a machine embodying at least one program of instructions executable by the machine to perform the capabilities of the present invention can be provided.

The flow diagrams depicted herein are just examples. There may be many variations to these diagrams or the steps (or operations) described therein without departing from the spirit of the invention. For instance, the steps may be performed in a differing order, or steps may be added, deleted or modified. All of these variations are considered a part of the claimed invention.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the following claims.

What is claimed is:

1. A computer-implemented method of processing genomic data comprising:
obtaining a first nucleotide locus and a second nucleotide locus representative of genomic data mapped to a genomic coordinate system;
performing correlation analysis on the first and second nucleotide loci, the performing including:
selecting a comparison type and a comparison value for use in performing the correlation analysis, the comparison type comprising one of intersection type or proximity type, and the comparison value comprising a number (n) of nucleotide positions, wherein n≧1, or a percentage number (pn) of nucleotide positions, wherein pn≧0, to be employed in determining whether the first nucleotide locus and the second nucleotide locus correlate,
comparing the first and second nucleotide loci for correlation, utilizing the selected comparison type and comparison value, wherein when intersection type is selected, and dependent on the correlation value selected, correlation is defined by the first nucleotide locus and the second nucleotide locus overlapping with at least the number (n) of nucleotide positions in common, or by the first nucleotide locus and the second nucleotide locus overlapping with at least the percent number (pn) of nucleotide positions in common relative to a smaller one of the first nucleotide locus and the second nucleotide locus, or when proximity type is selected, correlation is defined by the first nucleotide locus and the second nucleotide locus being within at least the number (n) of nucleotide positions, and
outputting results of the correlation analysis of the first and second nucleotide loci.

2. The method of claim 1, wherein the performing correlation analysis further comprises determining whether a first chromosome comprising the first nucleotide locus is before a second chromosome comprising the second nucleotide locus, and if so, providing an indication that the first nucleotide locus is before the second nucleotide locus, otherwise, determining whether the first chromosome is after the second chromosome, and if so, providing an indication that the first nucleotide locus is after the second nucleotide locus, otherwise, determining whether the first nucleotide locus is contained within the second nucleotide locus or the second nucleotide locus is contained within the first nucleotide locus, and if so, providing an indication that the first nucleotide locus and second nucleotide locus overlap, and if not, then performing the comparing of the first nucleotide locus and the second nucleotide locus using the selected comparison type and comparison value.

3. The method of claim 2, wherein the comparing further comprises temporarily adjusting a start coordinate and an end coordinate of the first nucleotide locus by the number (n) of nucleotide positions or the calculated percent number (pn) of nucleotide positions, and thereafter, determining whether the adjusted start position of the first nucleotide locus is after an end position of the second nucleotide locus, and if so, providing an indication that the first nucleotide locus is after the second nucleotide locus, otherwise, determining whether the adjusted end position of the first nucleotide locus is before a start position of the second nucleotide locus, and if so, providing an indication that the first nucleotide locus is before the second nucleotide locus, otherwise, providing an indication that the first nucleotide locus and the second nucleotide locus correlate.

4. The method of claim 1, wherein the performing correlation analysis further comprises identifying when the comparison value is a number (n) of nucleotide positions, and wherein when the comparison type is intersection type, the performing correlation analysis includes adjusting a start coordinate and an end coordinate of the first nucleotide locus by increasing the start coordinate of the first nucleotide locus by the number (n) of nucleotide positions and decreasing the end coordinate of the first nucleotide locus by the same number (n) of nucleotide positions to produce an adjusted start position and an adjusted end position for the first nucleotide locus, and wherein when the comparison type is proximity type, the performing correlation analysis includes adjusting a start coordinate and an end coordinate of the first nucleotide locus by decreasing the start coordinate of the first nucleotide locus by the number (n) of nucleotide positions and increasing the end coordinate of the first nucleotide locus by the same number (n) of nucleotide positions to produce an adjusted start position and an adjusted end position for the first nucleotide locus, wherein the comparing includes comparing the adjusted start position and the adjusted end position of the first nucleotide locus with a start coordinate and an end coordinate of the second nucleotide locus in determining whether the first nucleotide locus and the second nucleotide locus correlate.

5. The method of claim 1, wherein when the selected comparison value is a percentage number (pn), the performing correlation analysis further comprises identifying a size of the smaller one of the first nucleotide locus and the second nucleotide locus, and using the size and the percent number (pn) to identify a required number (x) of nucleotide positions to overlap for correlation to occur, and wherein the performing correlation analysis further comprises adjusting a start coordinate and an end coordinate of the first nucleotide locus by increasing the start coordinate of the first nucleotide locus by the required number (x) of nucleotide positions, and decreasing the end coordinate of the first nucleotide locus by the required number (x) of nucleotide positions to produce an adjusted start position and an adjusted end position of the first nucleotide locus, wherein the comparing includes comparing the adjusted start position and the adjusted end position of the first nucleotide locus with a start coordinate and an end coordinate of the second nucleotide locus in determining whether the first nucleotide locus and the second nucleotide locus correlate.

6. The method of claim 1, wherein selecting the comparison type and selecting the comparison value comprise pre-selecting by a user the comparison type and the comparison value.

7. The method of claim 1, further comprising initially obtaining a plurality of mapped data sets comprising genomic data mapped to the genomic coordinate system, and performing set correlation analysis of the plurality of mapped data sets to identify at a nucleotide level whether various nucleotide loci of the plurality of mapped data sets correlate, wherein the performing set correlation analysis comprises selecting the first nucleotide locus from a first mapped data set of the plurality of mapped data sets and selecting the second nucleotide locus from a second mapped data set of the plurality of mapped data sets, and after determining whether the first nucleotide locus and the second nucleotide locus correlate, repeating nucleotide loci selecting and correlation analysis for a plurality of nucleotide loci of the first mapped data set and second mapped data set, and outputting results of the set correlation analysis of the plurality of mapped data sets.

8. The method of claim 7, wherein the first mapped data set is a mapped experimental data set, and wherein obtaining the mapped experimental data set further comprises obtaining an experimental data set containing genomic data, and transforming the genomic data of the experimental data set to a chromosomal identification and a start coordinate and an end coordinate within the identified chromosome to produce the mapped experimental data set, and saving the mapped experimental data set in memory.

9. The method of claim 8, wherein the transforming comprises mapping data within the experimental data set to nucleotide loci, the nucleotide loci being represented as locus objects, each locus object further comprising logic to facilitate sorting and comparing of two or more locus objects of the experimental data set.

10. The method of claim 7, further comprising:
prior to performing set correlation analysis, ordering nucleotide loci within a mapped experimental data set of the plurality of mapped data sets relative to the genomic coordinate system to produce a set of ordered nucleotide loci;
automatically compressing the set of ordered nucleotide loci into a set of nucleotide regions, wherein two or more nucleotide loci which correlate are compressed into a single nucleotide region, and correlation is defined by intersection, with a nucleotide loci pair of the two or more nucleotide loci sharing at least one nucleotide position in common;
saving the set of nucleotide regions resulting from the automatically compressing; and
wherein performing set correlation analysis comprises performing set correlation analysis using the ordered, and compressed set as the first mapped data set and comparing each nucleotide region thereof with nucleotide loci or nucleotide regions within the second mapped data set of the plurality of mapped data sets.

11. The method of claim 10, wherein loci within each of the mapped data sets are ordered and compressed prior to performing set correlation analysis.

12. The method of claim 7, wherein performing set correlation analysis further comprises:
identifying and grouping at the nucleotide level correlated nucleotide loci of the first mapped data set and the second mapped data set;
for each group of correlated nucleotide loci, defining a data structure comprising a union locus extending across all correlated nucleotide loci within the group, and including the original nucleotide loci within the group which correlate; and
outputting the defined data structure, wherein the defined data structure with the union locus, and original nucleotide loci which correlate, functions as an accessible container for displaying, analyzing or retrieving of the information identified therein.

13. The method of claim 12, wherein the defining further comprises defining the data structure to include an intersection locus identifying nucleotide positions overlapping among the group of correlated nucleotide loci of the first mapped data set and the second mapped data set.

14. The method of claim 7, further comprising displaying a flow diagram of the processing, including a representation of the first mapped data set, the second mapped data set, the correlation analysis performed thereon, and the results of the correlation analysis thereof, the flow diagram allowing a user to interactively examine the first mapped data set, the second mapped data set, at least one parameter employed in the correlation analysis thereof, and the results of the correlation analysis.

15. The method of claim 1, further comprising obtaining a first locus object and a second locus object, the first locus object comprising the first nucleotide locus and the second locus object comprising the second nucleotide locus, and wherein each locus object comprises logic to facilitate the comparing of the first and second nucleotide loci, wherein the obtaining of the first and second locus objects further comprises obtaining at least one locus set object, the at least one locus set object comprising the first and second locus objects, and comprising logic to compress locus objects therein into locus regions to facilitate performing correlation analysis, and wherein at least one of the first and second nucleotide loci is represented as a locus region.

16. A system for processing genomic data comprising:
memory for holding a first nucleotide locus and a second nucleotide locus representative of genomic data mapped to a genomic coordinate system;
a correlation analysis tool to perform correlation analysis on the first nucleotide locus and the second nucleotide locus, the correlation analysis tool including:
select logic to designate a comparison type and a comparison value to be used in performing the correlation analysis, the comparison type comprising one of intersection type or proximity type, and the comparison value comprising a number (n) of nucleotide positions, wherein n≧1, or a percent number (pn) of nucleotide positions, wherein pn≧0, to be employed in determining whether the first nucleotide locus and the second nucleotide locus correlate,
comparison logic to determine whether the first and second nucleotide loci correlate, the comparison logic utilizing the selected comparison type and comparison value in performing the correlation analysis, wherein when intersection type is selected, and dependent on the correlation value selected, correlation is defined by the first nucleotide locus and the second nucleotide locus overlapping with at least the number (n) of nucleotide positions in common, or by the first nucleotide locus and the second nucleotide locus overlapping with at least the percent number (pn) of nucleotide positions in common relative to a smaller one of the first nucleotide locus and the second nucleotide locus, or when proximity type is selected, correlation is defined by the first nucleotide locus and the second nucleotide locus being within at least the number (n) of nucleotide positions, and
output logic to provide results of the correlation analysis of the first nucleotide locus and the second nucleotide locus.

17. The system of claim 16, wherein the memory holds a plurality of mapped data sets comprising genomic data mapped to the genomic coordinate system, and the correlation analysis tool performs set correlation analysis of the plurality of mapped data sets to identify at a nucleotide level whether various nucleotide loci of the plurality of mapped data sets correlate, wherein the correlation analysis tool comprises means for selecting the first nucleotide locus from a first mapped data set of the plurality of mapped data sets and means for selecting the second nucleotide locus from a second mapped data set of the plurality of mapped data sets, and after determining whether the first nucleotide locus and the second nucleotide locus correlate, means for repeating nucleotide loci selecting and correlation analysis for a plurality of nucleotide loci of the first mapped data set and second mapped data set, and means for outputting results of the set correlation analysis of the plurality of mapped data sets.

18. The system of claim 17, further comprising:
prior to performing set correlation analysis, means for ordering nucleotide loci within a mapped experimental data set of the plurality of mapped data sets relative to the genomic coordinate system to produce a set of ordered nucleotide loci;
means for automatically compressing the set of ordered nucleotide loci into a set of nucleotide regions, wherein two or more nucleotide loci which correlate are compressed into a single nucleotide region, and correlation is defined by intersection, with a nucleotide loci pair of the two or more nucleotide loci sharing at least one nucleotide position in common;
means for saving the set of nucleotide regions resulting from the automatically compressing; and
wherein the comparison logic performs set correlation analysis using the ordered, and compressed set as the first mapped data set and compares each nucleotide region thereof with nucleotide loci or nucleotide regions within the second mapped data set of the plurality of mapped data sets.

19. The system of claim 17, wherein the correlation analysis tool further comprises:
means for identifying and grouping at the nucleotide level correlated nucleotide loci of the first mapped data set and the second mapped data set;
for each group of correlated nucleotide loci, means for defining a data structure comprising a union locus extending across all correlated nucleotide loci within the group, and including the original nucleotide loci within the group which correlate, and an intersection locus identifying nucleotide positions overlapping among the group of correlated nucleotide loci of the first mapped data set and the second mapped data set; and
means for outputting the defined data structure, wherein the defined data structure with the union locus, and original nucleotide loci which correlate, functions as an accessible container for displaying, analyzing or retrieving of the information identified therein.

20. The system of claim 16, further comprising means for obtaining a first locus object and a second locus object, the first locus object comprising the first nucleotide locus and the second locus object comprising the second nucleotide locus, and wherein each locus object comprises logic to facilitate the comparing of the first and second nucleotide loci, and wherein the means for obtaining of the first and second locus objects further comprises means for obtaining at least one locus set object, the at least one locus set object comprising the first and second locus objects, and comprising logic to compress locus objects therein into locus regions to facilitate performing correlation analysis, and wherein at least one of the first and second nucleotide loci is represented as a locus region.

21. An article of manufacture comprising:
at least one computer-usable storage device comprising computer-readable program code logic to facilitate processing of genomic data, said computer-readable program code logic when executing performing the following:
obtaining a first nucleotide locus and a second nucleotide locus representative of genomic data mapped to a genomic coordinate system;
performing correlation analysis on the first nucleotide locus and second nucleotide locus, the performing including:
selecting a comparison type and a comparison value for use in performing the correlation analysis, the comparison type comprising one of intersection type or proximity type, and the comparison value comprising a number (n) of nucleotide positions, wherein n≧1, or a percentage number (pn) of nucleotide positions, wherein pn≧0, to be employed in determining whether the first nucleotide locus and the second nucleotide locus correlate; and
comparing the first and second nucleotide loci for correlation utilizing the selected comparison type and comparison value, wherein when intersection type is selected, and dependent on the correlation value selected, correlation is defined by the first nucleotide locus and the second nucleotide locus overlapping with at least the number (n) of nucleotide positions in common, or by the first nucleotide locus and the second nucleotide locus overlapping with at least the percent number (pn) of nucleotide positions in common relative to a smaller one of the first nucleotide locus and the second nucleotide locus, or when proximity type is selected, correlation is defined by the first nucleotide locus and the second nucleotide locus being within at least the number (n) of nucleotide positions, and
outputting results of the correlation analysis of the first and second nucleotide loci.

22. The article of manufacture of claim 21, wherein the computer-readable program code logic, when executing, further performs initially obtaining a plurality of mapped data sets comprising genomic data mapped to the genomic coordinate system, and performing set correlation analysis of the plurality of mapped data sets to identify at a nucleotide level whether various nucleotide loci of the plurality of mapped data sets correlate, wherein the performing set correlation analysis comprises selecting the first nucleotide locus from a first mapped data set of the plurality of mapped data sets and selecting the second nucleotide locus from a second mapped data set of the plurality of mapped data sets, and after determining whether the first nucleotide locus and the second nucleotide locus correlate, repeating nucleotide loci selecting and correlation analysis for a plurality of nucleotide loci of the first mapped data set and second mapped data set, and outputting results of the set correlation analysis of the plurality of mapped data sets.

23. The article of manufacture of claim 22, wherein the performing set correlation analysis further comprises:
identifying and grouping at the nucleotide level correlated nucleotide loci of the first mapped data set and the second mapped data set;
for each group of correlated nucleotide loci, defining a data structure comprising a union locus extending across all correlated nucleotide loci within the group, and including the original nucleotide loci within the group which correlate, and an intersection locus identifying nucleotide positions overlapping among the group of correlated nucleotide loci of the first mapped data set and the second mapped data set; and
outputting the defined data structure, wherein the defined data structure with the union locus, and original nucleotide loci which correlate, functions as an accessible container for displaying, analyzing or retrieving of the information identified therein.

24. The article of manufacture of claim 22, wherein the computer-readable program code logic, when executing, further performs displaying a flow diagram of the processing, including a representation of the first mapped data set, the second mapped data set, the correlation analysis performed thereon, and the results of the correlation analysis thereof, the flow diagram allowing a user to interactively examine the first mapped data set, the second mapped data set, at least one parameter employed in the correlation analysis thereof, and the results of the correlation analysis.

25. The article of manufacture of claim 21, wherein the computer-readable program code logic, when executing, further performs obtaining a first locus object and a second locus object, the first locus object comprising the first nucleotide locus and the second locus object comprising the second nucleotide locus, and wherein each locus object comprises logic to facilitate the comparing of the first and second nucleotide loci, and wherein the obtaining of the first and second locus objects further comprises obtaining at least one locus set object, the at least one locus set object comprising the first and second locus objects, and comprising logic to compress locus objects therein into locus regions to facilitate performing correlation analysis, and wherein at least one of the first and second nucleotide loci is represented as a locus region.
